(19) United States
(12) Patent Application Publication
(10) Pub. No.: US 2025/0249020 A1
(43) Pub. Date: Aug. 7, 2025

(54) COLLOIDALLY STABLE HEME-ALBUMIN COMPLEXES AND METHODS OF MAKING AND USING THEREOF

(71) Applicant: Ohio State Innovation Foundation, Columbus, OH (US)

(72) Inventors: Andre Palmer, Columbus, OH (US); Chintan Savla, Columbus, OH (US)

(21) Appl. No.: 18/856,949

(22) PCT Filed: Apr. 17, 2023
(86) PCT No.: PCT/US23/18808

§ 371 Date: Oct. 15, 2024

Related U.S. Application Data
(60) Provisional application No. 63/331,427, filed on Apr. 15, 2022.

(51) Int. Cl. A61K 31/555; A61K 38/00; A61K 45/06; A61K 47/60; A61K 47/64; C07K 1/34; C07K 14/76

(52) U.S. Cl. CPC A61K 31/555; A61K 45/06; A61K 47/60; A61K 47/643; C07K 1/34; C07K 14/76; A61K 38/00

(57) ABSTRACT

Disclosed herein are colloidally stable heme-albumin complexes, as well as compositions comprising these complexes, methods of making these complexes, and methods of using these complexes. These heme-albumin complexes can comprise from three to five heme molecules non-covalently associated with an albumin protein, and a plurality of hydrophilic polymer chains (e.g., polyethylene glycol (PEG) polymer chains) conjugated to the albumin protein.

### CROSS-REFERENCE TO RELATED APPLICATIONS

This application claims benefit of U.S. Provisional Application No. 63/331,427, filed Apr. 15, 2022, which is hereby incorporated herein by reference in its entirety.

### STATEMENT REGARDING FEDERALLY SPONSORED RESEARCH OR DEVELOPMENT

This invention was made with government support under grant numbers R01HL 126945, R01HL138116, R01HL 156526, R0 l HL158076, R01HL 159862, R01HL162120, and R01EB021926 awarded by the National Institutes of Health. The government has certain rights in the invention.

### BACKGROUND

The effects of free heme on the innate immune system following exposure to cell-free hemoglobin (Hb) has been studied. However, a recent study challenged the assumption that heme is a major pro-inflammatory mediator in vito by demonstrating that protein-associated heme or Hb did not induce inflammatory gene expression over a broad range of exposure conditions, which was corroborated in human endothelial cell culture as well as mouse macrophage cell culture. Other studies observed that heme elicits anti-inflammatory effects by promoting overexpression of heme-oxygenase 1 (HO-1), which can catalyze the breakdown of heme into biliverdin, iron and carbon monoxide. However, to harness the anti-inflammatory properties of heme for therapeutic applications, improved heme-containing compositions are needed.

### SUMMARY

Heme is a highly hydrophobic molecule, and is mainly soluble in organic solvents or basic aqueous solutions. Therefore, in order to increase heme's aqueous solubility, colloidal stability, and anti-inflammatory therapeutic potential, heme was non-covalently associated with the ubiquitous plasma protein human serum albumin (HSA) to form a heme-albumin complex. Further, hydrophilic polymer chains (e.g., poly(ethylene glycol) (PEG) chains) were conjugated to the surface of the heme-albumin complex. This strategy first increases the aqueous solubility of heme by first binding it to a carrier protein (HSA) that itself is soluble in aqueous solution, and then subsequently further improves the colloidal stability of heme-albumin complex by, for example, modifying the heme-albumin complex with one or more hydrophilic polymer chains.

In some examples, the colloidally stable heme-albumin complex can be formed by first complexing the heme with albumin to form a heme-albumin complex, and then conjugating one or more hydrophilic polymer chains (e.g., PEG chains) to the surface of the heme-albumin complex to form a colloidally stable heme-albumin complex. For example, provided herein are methods for forming a colloidally stable heme-albumin complex that comprise contacting albumin with heme under conditions effective to form a heme-albumin complex; conjugating one or more hydrophilic polymers (e.g., one or more polyethylene glycol (PEG) polymers) to the heme-albumin complex to form a crude colloidally stable heme-albumin complex; and filtering the crude colloidally stable heme-albumin complex by ultrafiltration against a filtration membrane, thereby forming a retentate fraction comprising the colloidally stable heme-albumin complex and a permeate fraction comprising low molecular weight contaminants.

In some embodiments, the colloidally stable heme-albumin complex can be substantially free of free heme, aggregated heme particulates, unconjugated hydrophilic polymers, and other low molecular weight contaminants. In certain embodiments, the colloidally stable heme-albumin complex can comprise less than 1% by weight (e.g., less than 0.5%, or less than 0.1%) free heme, aggregated heme particulates, unconjugated hydrophilic polymers, and other low molecular weight contaminants having a molecular weight of less than 50 kDa.

In some examples, contacting albumin with heme under conditions effective to form a heme-albumin complex can comprise incubating the albumin with heme at a pH of 8 or more, such as a pH of from 8 to 10. In some examples, the heme and the albumin are present in the contacting step at a molar ratio of heme:albumin of at least 5:1, such as a molar ratio of from 5:1 to 15:1, such as from 5:1 to 12:1, from 5:1 to 10:1, or from 5:1 to 8:1. In some examples, contacting albumin with heme under conditions effective to form a heme-albumin complex can further comprise neutralizing the heme-albumin complex.

In some examples, the filtration membrane can have a filter rated for removing solutes having a molecular weight of from 1 to 50 kDa. In some examples, filtering the crude colloidally stable heme-albumin complex by ultrafiltration can comprise filtering for at least 3 diafiltrations cycles, such as at least 4 diafiltration cycles, at least 5 diafiltration cycles, at least 6 diafiltration cycles, at least 7 diafiltrations cycle, at least 8 diafiltration cycles, at least 9 diafiltrations cycle, or at least 10 diafiltration cycles. In certain embodiments, the ultrafiltration can comprise tangential flow filtration.

In some examples, the one or more hydrophilic polymers can comprise poly(lactide), poly(glycolide), a poly(orthoesters), a poly(caprolactone), polylysine, poly(ethylene imine), poly(acrylic acid), a poly(urethane), a poly(anhydride), a poly(ester), poly(trimethylene carbonate), poly(ethyleneimine), a poly(acrylic acid), a poly(urethane), a poly(beta amino ester), a poly(alkylene oxide) such as polyethylene glycol (PEG), a zwitterionic polymer, a copolymer thereof, or a blend thereof.

In some examples, conjugating the one or more hydrophilic polymers (e.g., one or more polyethylene glycol (PEG) polymers) to the heme-albumin complex to form the crude PEGylated heme-albumin complex can comprise contacting the heme-albumin complex with a derivatized hydrophilic polymer under conditions permitting formation of a covalent bond between the hydrophilic polymer and the heme-albumin complex so as to form the crude colloidally stable heme-albumin complex.

In certain examples, the one or more hydrophilic polymers can comprise polyethylene glycol (PEG). In some of these examples, conjugating the one or more PEG polymers to the heme-albumin complex to form the crude colloidally stable heme-albumin complex comprises contacting the heme-albumin complex with a derivatized PEG polymer under conditions permitting formation of a covalent bond between the PEG polymer and the heme-albumin complex so as to form the crude colloidally stable heme-albumin complex. The derivatized PEG can comprise, for example, succinimidyl-PEG, cyanuric chloride-PEG, or maleimide-PEG. In some of these examples, conjugating the one or more PEG polymers to the heme-albumin complex to form the crude colloidally stable heme-albumin complex further comprises contacting the heme-albumin complex with a thiolation reagent, such as 2-iminothiolane hydrochloride, to introduce thiol moieties that react with the derivatized PEG.

In some examples, the one or more hydrophilic polymers (e.g., PEG) can each have a molecular weight of from 200 Da to 20,000 Da, such as from 1,000 Da to 10,000 Da, or from 3,000 Da to 6,000 Da, or about 5,000 Da.

In some examples, the method can further comprise filtering the heme-albumin complex by ultrafiltration against a filtration membrane prior to conjugating one or more hydrophilic polymers to the heme-albumin complex, thereby forming a retentate fraction comprising the heme-albumin complex and a permeate fraction comprising low molecular weight contaminants. In some examples, following filtration, the heme-albumin complex is substantially free of free heme, aggregated heme particulates, and other low molecular weight contaminants. For example, in some embodiments, following filtration, the heme-albumin complex comprises less than 1% by weight (e.g., less than 0.5% by weight, or less than 0.1% by weight) free heme, aggregated heme particulates, and other low molecular weight contaminants having a molecular weight of less than 50 kDa.

In some examples, the colloidally stable heme-albumin complex comprises five heme molecules associated with one albumin protein.

In some examples, the colloidally stable heme-albumin complex is stable in aqueous solution after 72 hours of storage at 4° C. without loss or precipitation of heme.

In some examples, the colloidally stable heme-albumin complex can exhibit a hydrodynamic diameter of from about 10 nm to about 15 nm, as determined by dynamic light scattering (DLS).

In some examples, the colloidally stable heme-albumin complex can comprise from 3 to 10 hydrophilic polymer chains (e.g., PEG chains) conjugated to the heme-albumin complex, as determined by thiol quantification.

In some examples, the albumin comprises a serum albumin, such as human serum albumin or recombinant human serum albumin.

In other examples, the colloidally stable heme-albumin complex can be formed by first conjugating one or more hydrophilic polymer chains (e.g., PEG chains) to the surface of the albumin to form polymer-modified albumin, and then complexing the heme with the polymer-modified albumin to form a colloidally stable heme-albumin complex. For example, also provided are method for forming a colloidally stable heme-albumin complex that comprise conjugating one or more hydrophilic polymers (e.g., one or more polyethylene glycol (PEG) polymers) to albumin to form polymer-modified albumin; contacting the polymer-modified albumin with heme under conditions effective to form a crude colloidally stable heme-albumin complex; and filtering the crude colloidally stable heme-albumin complex by ultrafiltration against a filtration membrane, thereby forming a retentate fraction comprising the colloidally stable heme-albumin complex and a permeate fraction comprising low molecular weight contaminants.

In some embodiments, the colloidally stable heme-albumin complex can be substantially free of free heme, aggregated heme particulates, unconjugated hydrophilic polymers, and other low molecular weight contaminants. In certain embodiments, the PEGylated heme-albumin complex can comprise less than 1% by weight (e.g., less than 0.5%, or less than 0.1%) free heme, aggregated heme particulates, unconjugated hydrophilic polymers, and other low molecular weight contaminants having a molecular weight of less than 50 kDa.

In some examples, contacting the polymer-modified albumin with heme under conditions effective to form a crude colloidally stable heme-albumin complex can comprise incubating the polymer-modified albumin with heme at a pH of 8 or more, such as a pH of from 8 to 10. In some examples, the heme and the polymer-modified albumin are present in the contacting step at a molar ratio of heme:polymer-modified albumin of at least 5:1, such as a molar ratio of from 5:1 to 15:1, such as from 5:1 to 12:1, from 5:1 to 10:1, or from 5:1 to 8:1. In some examples, contacting polymer-modified albumin with heme under conditions effective to form a crude colloidally stable heme-albumin complex further comprises neutralizing the crude colloidally stable heme-albumin complex.

In some examples, the filtration membrane can have a filter rated for removing solutes having a molecular weight of from 1 to 50 kDa. In some examples, filtering the crude colloidally stable heme-albumin complex by ultrafiltration can comprise filtering for at least 3 diafiltrations cycles, such as at least 4 diafiltration cycles, at least 5 diafiltration cycles, at least 6 diafiltration cycles, at least 7 diafiltrations cycle, at least 8 diafiltration cycles, at least 9 diafiltrations cycle, or at least 10 diafiltration cycles. In certain embodiments, the ultrafiltration can comprise tangential flow filtration.

In some examples, the one or more hydrophilic polymers can comprise poly(lactide), poly(glycolide), a poly(orthoesters), a poly(caprolactone), polylysine, poly(ethylene imine), poly(acrylic acid), a poly(urethane), a poly(anhydride), a poly(ester), poly(trimethylene carbonate), poly(ethyleneimine), a poly(acrylic acid), a poly(urethane), a poly(beta amino ester), a poly(alkylene oxide) such as polyethylene glycol (PEG), a zwitterionic polymer, a copolymer thereof, or a blend thereof.

In some examples, conjugating the one or more hydrophilic polymers (e.g., one or more polyethylene glycol (PEG) polymers) to the albumin to form the polymer-modified albumin can comprise contacting the albumin with a derivatized hydrophilic polymer under conditions permitting formation of a covalent bond between the hydrophilic polymer and the albumin so as to form the polymer-modified albumin.

In certain examples, the one or more hydrophilic polymers can comprise polyethylene glycol (PEG). In some of these examples, conjugating the one or more PEG polymers to the albumin to form PEGylated albumin comprises contacting the albumin with a derivatized PEG polymer under conditions permitting formation of a covalent bond between the PEG polymer and the albumin so as to form the PEGylated albumin. The derivatized PEG can comprise, for example, succinimidyl-PEG, cyanuric chloride-PEG, or maleimide-PEG. In some of these examples, conjugating the one or more PEG polymers to the albumin to form the PEGylated albumin further comprises contacting the albumin with a thiolation reagent, such as 2-iminothiolane hydrochloride, to introduce thiol moieties that react with the derivatized PEG.

In some examples, the one or more hydrophilic polymers (e.g., PEG) can each have a molecular weight of from 200 Da to 20,000 Da, such as from 1,000 Da to 10,000 Da, or from 3,000 Da to 6,000 Da, or about 5,000 Da.

In some examples, the method further comprises filtering the polymer-modified albumin by ultrafiltration against a filtration membrane prior to contacting the polymer-modified albumin with heme under conditions effective to form a crude colloidally stable heme-albumin complex, thereby forming a retentate fraction comprising the polymer-modified albumin and a permeate fraction comprising low molecular weight contaminants. In some examples, following filtration, the polymer-modified albumin is substantially free of unconjugated hydrophilic polymers and other low molecular weight contaminants. For example, following filtration, the polymer-modified albumin comprises less than 1% by weight (e.g., less than 0.5% by weight, or less than 0.1% by weight) free unconjugated hydrophilic polymers and other low molecular weight contaminants having a molecular weight of less than 50 kDa.

In some examples, the colloidally stable heme-albumin complex comprises five heme molecules associated with one albumin protein.

In some examples, the colloidally stable heme-albumin complex is stable in aqueous solution after 72 hours of storage at 4° C. without loss or precipitation of heme.

In some examples, the colloidally stable heme-albumin complex can exhibit a hydrodynamic diameter of from about 10 nm to about 15 nm, as determined by dynamic light scattering (DLS).

In some examples, the colloidally stable heme-albumin complex can comprise from 3 to 10 hydrophilic polymer chains (e.g., PEG chains) conjugated to the heme-albumin complex, as determined by thiol quantification.

In some examples, the albumin comprises a serum albumin, such as human serum albumin or recombinant human serum albumin.

Also provided are colloidally stable heme-albumin complexes prepared by the methods described herein, as well as compositions that comprise these complexes dissolved or dispersed in an aqueous carrier.

In some examples, the colloidally stable heme-albumin complex can comprise from three to six heme molecules non-covalently associated with an albumin protein, and a plurality of hydrophilic polymers (e.g., polyethylene glycol (PEG) polymers) conjugated to the albumin protein.

The composition can be substantially free of free heme, aggregated heme particulates, unconjugated hydrophilic polymers, and other low molecular weight contaminants. For example, in some embodiments, the composition can comprise less than 1% by weight (e.g., less than 0.5% by weight, or less than 0.1% by weight) free heme, aggregated heme particulates, unconjugated hydrophilic polymers, and other low molecular weight contaminants having a molecular weight of less than 50 kDa.

In some examples, the one or more hydrophilic polymers can comprise poly(lactide), poly(glycolide), a poly(orthoesters), a poly(caprolactone), polylysine, poly(ethylene imine), poly(acrylic acid), a poly(urethane), a poly(anhydride), a poly(ester), poly(trimethylene carbonate), poly(ethyleneimine), a poly(acrylic acid), a poly(urethane), a poly(beta amino ester), a poly(alkylene oxide) such as polyethylene glycol (PEG), a zwitterionic polymer, a copolymer thereof, or a blend thereof. In certain examples, the one or more hydrophilic polymers can comprise polyethylene glycol (PEG).

In some examples, the one or more hydrophilic polymers (e.g., PEG) can each have a molecular weight of from 200 Da to 20,000 Da, such as from 1,000 Da to 10,000 Da, or from 3,000 Da to 6,000 Da, or about 5,000 Da.

In some examples, the colloidally stable heme-albumin complex comprises five heme molecules associated with one albumin protein.

In some examples, the colloidally stable heme-albumin complex is stable in aqueous solution after 72 hours of storage at 4° C. without loss or precipitation of heme.

In some examples, the colloidally stable heme-albumin complex exhibits a hydrodynamic diameter of from about 10 nm to about 15 nm, as determined by dynamic light scattering (DLS).

In some examples, the colloidally stable heme-albumin complex comprises from 3 to 10 hydrophilic polymer chains conjugated to the heme-albumin complex, as determined by thiol quantification.

In some examples, the albumin comprises a serum albumin, such as human serum albumin or recombinant human serum albumin.

Also provided are methods of using the colloidally stable heme-albumin complexes described herein, for example, in therapeutic applications. For example, provided herein are methods of reducing inflammation in a subject that comprise administering a therapeutically effective amount of a colloidally stable heme-albumin complex described herein.

Also provided are methods of treating an ophthalmological disorder in a subject in need thereof comprising contacting the eye of the subject a therapeutically effective amount of a composition comprising a colloidally stable heme-albumin complex described herein.

In some embodiments, the composition further comprises an additional active agent. In some examples, the additional active agent can comprise an ophthalmic drug, such as an anti-glaucoma agent, an anti-angiogenesis agent, an anti-vascular endothelial growth factor (VEGF) agent, an anti-infective agent, an anti-inflammatory agent, a growth factor, an immunosuppressant agent, an anti-allergic agent, or any combinations thereof.

The ophthalmological disorder can comprise, for example, acute macular neuroretinopathy; Behcet's disease; neovascularization, including choroidal neovascularization; diabetic uveitis; histoplasmosis; infections, such as fungal or viral-caused infections; macular degeneration, such as acute macular degeneration (AMD), including wet AMD, non-exudative AMD and exudative AMD; retinal degenerative diseases such as geographic atrophy; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic ophthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, nonretinopathy diabetic retinal dysfunction, retinitis pigmentosa, a cancer, and glaucoma.

In certain embodiments, the ophthalmological disorder can be AMD, such as dry AMD.

In some embodiments, contacting the eye of the subject can comprise topically applying the composition to the eye of the subject. In other embodiments, contacting the eye of the subject can comprise injecting the composition into the eye of the subject (e.g., injecting the composition into the vitreous chamber of the eye). In some embodiments, injecting into the eye of the subject comprises an intravitreal injection, a subconjunctival injection, a subtenon injection, a retrobulbar injection, or a suprachoroidal injection.

### DESCRIPTION OF DRAWINGS

FIG. 1 is a schematic illustrating heme incorporation into HSA and subsequent PEGylation of the protein. Heme was selectively and non-selectively bound to HSA, and the PEG chains were attached to primary amines (lysine residues) via thiol-maleimide chemistry.

FIGS. 2A-2C show the SEC-HPLC analysis of HSA, heme-HSA, PEG-HSA, PEGylated heme-HSA (PEG-heme-HSA), and heme incorporated PEG-HSA (heme-PEG-HSA). FIG. 2A is a plot showing the normalized absorbance at 280 nm (detects total protein). FIG. 2B is a plot showing the absorbance at 398 nm (detects heme). FIG. 2C is a plot showing the emission spectra at 330 nm after excitation at 285 nm (heme incorporation quenches protein fluorescence).

FIG. 3A is a plot showing the UV-visible absorbance spectra of heme-NaOH, HSA, heme-HSA, and PEG-heme-HSA samples.

FIG. 3B is a plot detailing the quantification of heme not bound to HSA. After exceeding a 6:1 heme:HSA molar ratio, the size of the heme pellet significantly increased in mass denoting saturation of the heme binding sites (specific and non-specific) in HSA.

FIG. 4 shows the dynamic light scattering (DLS) analysis of HSA and heme-incorporated derivatives. PEGylation resulted in an increase in hydrodynamic diameter of HSA and heme-HSA.

FIGS. 5A-5B show the SEC-HPLC analysis of (FIG. 5A) HSA and (FIG. 5B) HSA thawed from −80° C. storage, and subsequently stored at 4° C. for 0, 24, and 72 hours. All samples were stable during 72 hours of storage, however there was HSA dimer formation after HSA was exposed to a freeze-thaw cycle.

FIG. 5C shows the SEC-HPLC analysis of HSA samples after undergoing a pH shift from 7.4 to 8.6 and rebalancing to 7.4. No change in the quaternary structure was observed throughout the pH balancing process.

FIGS. 6A-6D are plots evaluating the storage stability of heme-HSA and PEG-heme-HSA. FIG. 6A and FIG. 6C show the SEC-HPLC analysis of heme-HSA and PEG-heme-HSA stored at 4° C. for 0, 24, and 72 hours. FIG. 6B and FIG. 6D SEC-HPLC analysis of heme-HSA and PEG-heme-HSA samples frozen at −80° C., thawed at 4° C., and stored for 0, 24, and 72 hours at 4° C. PEGylated samples demonstrated higher colloidal stability and no change in quaternary structure during 72-hour storage, whereas unPEGylated heme-HSA showed dimer formation after 24 hours in storage and after exposure to a single freeze thaw cycle.

FIGS. 7A-7C show CD analysis of HSA (FIG. 7A), heme-HSA intermediates (FIG. 7B), and PEG-heme-HSA (FIG. 7C). Samples were collected at different points during the synthesis process to monitor the α-helical content of HSA. Intermediate 1—Heme-NaOH+HSA start of reaction pH 8.5; Intermediate 2—Heme-NaOH+HSA end of reaction pH 8.6; Intermediate 3—Heme-NaOH+HSA pH balanced to 7.4; heme-HSA final product pH 7.4 after diafiltration; PEG-heme-HSA final product pH 7.4 after diafiltration.

FIGS. 8A-8C show the MADLI-TOF MS analysis of HSA (FIG. 8A) and PEG-heme-HSA (FIG. 8B and FIG. 8C) samples. HSA showed a characteristic single-charged peak at ˜66,000 m/z along with +2 and +3 charged species. Post-PEGylation, the single charged species shifted to ˜80,000 m/z denoting successful surface conjugation. A 616 m/z peak was also observed denoting the presence of heme and its' successful incorporation into HSA.

### DETAILED DESCRIPTION

The present invention can be understood more readily by reference to the following detailed description, examples, drawings, and claims, and their previous and following description. However, before the present articles, systems, and/or methods are disclosed and described, it is to be understood that this invention is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. To this end, those skilled in the relevant art will recognize and appreciate that many changes can be made to the various aspects of the invention described herein, while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

### Definitions

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable subcombination.

As used in the description and the appended claims, the singular forms “a,” “an,” and “the” include plural referents unless the context clearly dictates otherwise.

As used herein, the terms “optional” or “optionally” mean that the subsequently described event or circumstance may or may not occur and that the description includes instances where said event or circumstance occurs and instances where it does not.

It is also to be understood that the terminology used herein is for the purpose of describing particular aspects only and is not intended to be limiting. As used in the specification and in the claims, the term “comprising” can include the aspects “consisting of” and “consisting essentially of.” Unless defined otherwise, all technical and scientific terms used herein have the same meaning as commonly understood by one of ordinary skill in the art to which this invention belongs. In this specification and in the claims, which follow, reference will be made to a number of terms that shall be defined herein.

For the terms “for example” and “such as,” and grammatical equivalences thereof, the phrase “and without limitation” is understood to follow unless explicitly stated otherwise.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value and/or to “about” another particular value. When such a range is expressed, another aspect includes from the one particular value and/or to the other particular value.

Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Unless stated otherwise, the term “about” means within 5% (e.g., within 2% or 1%) of the particular value modified by the term “about.”

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

It will be understood that, although the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or a section. Thus, a first element, component, region, layer, or section discussed below could be termed a second element, component, region, layer, or section without departing from the teachings of example embodiments.

As used herein, the term “substantially” means that the subsequently described event or circumstance completely occurs or that the subsequently described event or circumstance generally, typically, or approximately occurs.

As used herein, the term “substantially,” in, for example, the context “substantially identical” or “substantially similar” refers to a method or a system, or a component that is at least about 80%, at least about 85%, at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

It is understood that in aspects disclosed herein, the terms “diacycle” and “diafiltration cycles” are used interchangeably.

As used herein, the term “ultrafiltration” is used for processes and systems employing membranes rated for removing solutes having a molecular weight between about 1 kDa and 1000 kDa.

As used herein, the term “tangential-flow filtration” refers to a process in which the fluid mixture containing the components to be separated by filtration is recirculated at velocities tangential to the plane of the filtration membrane to reduce fouling of the filter. In such filtrations a pressure differential is applied along the length of the filtration membrane to cause the fluid and filterable solutes to flow through the membrane (i.e., filter). This filtration is suitably conducted as a batch process as well as a continuous-flow process. For example, the solution may be passed repeatedly over the membrane while that fluid which passes through the filter is continually drawn off into a separate unit or the solution is passed once over the membrane and the fluid passing through the filter is continually processed downstream.

As used herein, the term “subject” refers to an animal, for example a human, to whom treatment, including prophylactic treatment, with a composition as disclosed herein, is provided. The term “subject” as used herein refers to human and non-human animals. The term “non-human animals” and “non-human mammals” are used interchangeably herein and includes all vertebrates, e.g., mammals, such as non-human primates, (particularly higher primates), sheep, dogs, rodents (e.g., mouse or rat), guinea pigs, goats, pigs, cats, rabbits, cows, horses, and non-mammals such as chickens, amphibians, reptiles etc. In one embodiment, the subject is human. In another embodiment, the subject is an experimental animal or animal substitute as a disease model. In another embodiment, the subject can comprise a pet or companion animal, such as a dog or cat.

The term “heme” as used herein refers to a prosthetic group comprising an iron atom in the center of a large organic cyclic macromolecule called porphyrin.

Ranges of values defined herein include all values within the range as well as all sub-ranges within the range. For example, if the range is defined as an integer from 0 to 10, the range encompasses all integers within the range and any and all subranges within the range, e.g., 1-10, 1-6, 2-8, 3-7, 3-9, etc.

While aspects of the present invention can be described and claimed in a particular statutory class, such as the system statutory class, this is for convenience only, and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to arrangement of steps or operational flow, plain meaning derived from grammatical organization or punctuation, or the number or type of aspects described in the specification.

The present invention may be understood more readily by reference to the following detailed description of various aspects of the invention and the examples included therein and to the Figures and their previous and following description.

### Colloidally Stable Heme-Albumin Complexes

Provided are colloidally stable heme-albumin complexes. The colloidally stable heme-albumin complexes can comprise from three to six heme molecules (e.g., three heme molecules, four heme molecules, five heme molecules, or six heme molecules) non-covalently associated with an albumin protein. In some examples, the colloidally stable heme-albumin complex comprises five heme molecules associated with one albumin protein. In some examples, the colloidally stable heme-albumin complex comprises six heme molecules associated with one albumin protein.

The complex further comprises a plurality of hydrophilic polymer chains (e.g., PEG chains) conjugated to the albumin protein. A hydrophilic polymer is one generally that attracts water, as compared to a hydrophobic polymer which generally repels water. A hydrophilic or a hydrophobic polymer can be identified, for example, by preparing a sample of the polymer and measuring its contact angle with water (typically, the polymer will have a contact angle of less than 60°,

Examples of hydrophilic polymers include, but are not limited to, poly(lactide) (or polylactic acid)), poly(glycolide) (or poly(glycolic acid)), poly(orthoesters), poly(caprolactones), polylysine, poly(ethylene imine), poly(alkylene oxides), poly(acrylic acid), poly(urethanes), poly(anhydrides), poly(esters), poly(trimethylene carbonate), poly(ethyleneimine), poly(acrylic acid), poly(urethane), poly(beta amino esters), poly(alkylene oxide), or the like, and copolymers or derivatives of these and/or other polymers, for example, poly(lactide-co-glycolide) (PLGA).

In some examples, the hydrophilic polymer can comprise a zwitterionic polymer. Examples of zwitterionic polymers include poly(sulfobetaines), poly(carboxybetaines), poly(phosphobetaines), copolymers thereof and the like. Specific examples of zwitterionic polymers include poly(3-[N-(2-acrylamidoethyl)dimethylammonio]propanesulfonate) poly(3-[N-(2-methylacrylamidoethyl)dimethylammonio]propanesulfonate), poly(3-[N-(2-methacryloxyethyl)dimethylammonio]propanesulfonate, poly(3-(N,N-dimethyl-N-(4-vinyl-phenyl)-ammonio)propanesulfonate, poly(3-[N-(2-acrylamidoethyl)dimethylammonio]propionate), poly(3-[N-(2-methylacrylamidoethyl)dimethylammonio]propionate), poly(3-[N-(2-methacryloxyethyl)dimethylammonio]propionte, poly(3-(N,N-dimethyl-N-(4-vinyl-phenyl)-ammonio)propionate, and poly(2-methacryloyloxyethyl phosphorylcholine.

In some examples, the hydrophilic polymer can comprise a poly(alkylene glycol) (also known as poly(alkylene oxide)), such as polypropylene glycol), or poly(ethylene oxide), also known as poly(ethylene glycol) (“PEG”), having the formula —(CH₂—CH₂—O)ᵤ—, where n is any positive integer. As used herein, the term “PEGylation” generally refers to linking to polyethylene glycol (PEG). Thus, “PEGylated” albumin refers to an albumin that has PEG conjugated (e.g., covalently bonded) to it.

In some examples, the one or more hydrophilic polymers each have a molecular weight of from 200 Da to 20,000 Da, such as from 1,000 Da to 10,000 Da, or from 3,000 Da to 6,000 Da, or about 5,000 Da.

In some examples, the colloidally stable heme-albumin complex comprises from 3 to 10 hydrophilic polymer chains (e.g., PEG chains) conjugated to the heme-albumin complex, as determined by thiol quantification.

The composition can be substantially free of free heme, aggregated heme particulates, unconjugated hydrophilic polymers, and other low molecular weight contaminants. For example, in some embodiments, the composition can comprise less than 1% by weight (e.g., less than 0.5% by weight, or less than 0.1% by weight) free heme, aggregated heme particulates, unconjugated hydrophilic polymers, and other low molecular weight contaminants having a molecular weight of less than 50 kDa.

In some examples, the albumin comprises a serum albumin, such as human serum albumin. Human serum albumin (HSA), is a 65 kDa non-glycosylated chain. Circulating at approximately 35-55 mg/mL in plasma, HSA is the most abundant plasma protein with a diverse set of functions. For example, HSA regulates oncotic pressure, binds and transports a variety of endogenous and exogenous molecules, and can have enzymatic and antioxidant properties (see Ascenzi, P., di Masi, A., Fanali, G. & Fasano, M. Heme-based catalytic properties of human serum albumin. Cell death Discov. 1, 15025 (2015)). Clinical applications of albumin include, blood volume replacement, emergency shock treatment, treatment of burns and other cases of hypovolemia (see Taverna, M., Marie, A.-L., Mira, J.-P. & Guidet, B. Specific antioxidant properties of human serum albumin. Ann. Intensive Care 3, 4 (2013)). The benefits from HSA infusion have been largely attributed to its antioxidant properties. Its high concentration in plasma and free thiol group (Cys34 residue) accounts for 80% of thiols in plasma, making HSA the major extracellular source of free thiols. The Cys34 residue can scavenge various free radicals (HSA accounts for 70% of plasma free-radical trapping) involved in the damaging oxidative pathways of hemolysis such as hydrogen peroxide, peroxynitrite, and superoxide (see Buehler, P. W., D'Agnillo, F. & Schaer, D. J. Hemoglobin-based oxygen carriers: from mechanisms of toxicity and clearance to rational drug design. Trends Mol. Med. 16, 447-457 (2010)).

Moreover, HSA can bind to both free heme and free iron (see Loban, A., Kime, R. & Powers, H. Iron-binding antioxidant potential of plasma albumin. Clin. Sci. (Lond). 93, 445-51 (1997)). Although at a lower affinity than Hpx (K_{d}≈10 nM), heme binding to HSA decreases free heme-mediated oxidative damage. Furthermore, although at an equimolar heme concentration, HSA does not fully prevent lipid oxidation, at concentrations of 4×molar excess, HSA has been shown to prevent heme oxidative damage (see Miller, Y. I., Felikman, Y. & Shaklai, N. The involvement of low-density lipoprotein in hemin transport potentiates peroxidative damage. Biochim. Biophys. Acta—Mol. Basis Dis. 1272, 119-127 (1995)). Yet, unlike Hpx, HSA itself is prone to oxidation due to the bound heme. Similarly, the iron binding properties of HSA prevents oxidative damage due to free iron. Furthermore, hemoglobin (Hb) mediated lipid peroxidation can be prevented via HSA administration. Thus, HSA can function as an iron and heme transport/carrier. Furthermore, kinetic studies have revealed that, upon heme release from Hb, the majority of heme is initially bound by low- and high-density lipoproteins (see Miller, Y. I. & Shaklai, N. Kinetics of hemin distribution in plasma reveals its role in lipoprotein oxidation. Biochim. Biophys. Acta 1454, 153-64 (1999)). Heme is then transferred first to albumin which then transports the heme to Hpx. Finally, the same mechanism of heme transport via albumin has been shown with respect to heme bound to the RBC membrane.

Another interesting property of HSA related to hemolysis is its role in bilirubin transport. In addition to aiding in transport of catabolized heme, given the role of bilirubin as a physiological reductant, bilirubin-bound HSA can have enhanced antioxidant properties by preventing lipid peroxidation (see Neuzil, J. & Stocker, R. Free and albumin-bound bilirubin are efficient co-antioxidants for alpha-tocopherol, inhibiting plasma and low-density lipoprotein lipid peroxidation. J. Biol. Chem. 269, 16712-9 (1994)).

Interestingly conditions in which administration of HSA is clinically recommended may benefit from hemolysis treatment proteins. For example, during severe burns, septic shock, organ transplantation, or surgeries HSA can be administered as a plasma expander (see Liumbruno, G., Bennardello, F., Lattanzio, A., Piccoli, P. & Rossettias, G. Recommendations for the use of albumin and immunoglobulins. Blood Tansfus. 7, 216 (2009)). Yet, these conditions have also been shown to have hemolytic traits (see Effenberger-Neidnicht, K. & Hartmann, M. Mechanisms of Hemolysis During Sepsis. Inflammation 41, 1569-1581 (2018); Vermeulen Windsant, I. C. et al. Hemolysis during cardiac surgery is associated with increased intravascular nitric oxide consumption and perioperative kidney and intestinal tissue damage. Front. Physiol. 5, 340 (2014); Achkar, R., Chiba, A. K., Zampieri-Filho, J. P., Pestana, J. O. M. & Bordin, J. O. Hemolytic anemia after kidney transplantation: a prospective analysis. Transfusion 51, 2495-2499 (2011); Achkar, R., Chiba, A. K., Zampieri-Filho, J. P., Pestana, J. O. M. & Bordin, J. O. Hemolytic anemia after kidney transplantation: a prospective analysis. Transfusion 51, 2495-2499 (2011); Petz, L. D. Immune hemolysis associated with transplantation. Semin. Hematol. 42, 145-55 (2005); Norman, T. E. et al. Intravascular hemolysis associated with severe cutaneous burn injuries in five horses. J. Am. Vet. Med. Assoc. 226, 2039-43, 2002 (2005); and Endoh, Y., Kawakami, M., Orringer, E. P., Peterson, H. D. & Meyer, A. A. Causes and time course of acute hemolysis after burn injury in the rat. J. Burn Care Rehabil. 13, 203-9). Thus, administration of a protein cocktail containing both HSA and hemolysis scavenging proteins could yield better patient outcomes.

Studies have demonstrated a receptor for heme which can explain heme delivery via heme-albumin (see Taketani, S. Aquisition, Mobilization and Utilization of Cellular Iron and Heme: Endless Findings and Growing Evidence of Tight Regulation. Tohoku J. Exp. Med. 205, 297-318 (2005); Worthington, M. T., Cohn, S. M., Miller, S. K., Luo, R. Q. & Berg, C. L. Characterization of a human plasma membrane heme transporter in intestinal and hepatocyte cell lines. Am. J. Physiol. Liver Physiol. 280, G1172-G1177 (2001); Noyer, C. M., Immenschuh, S., Liem, H. H., Muller-Eberhard, U. & Wolkoff, A. W. Initial heme uptake from albumin by short-term cultured rat hepatocytes is mediated by a transport mechanism differing from that of other organic anions. Hepaltology 28, 150-155 (1998); Taketani, S. et al. Furthermore, both heme and heme-albumin have been shown to have similar heme oxygenase-1 (HO-1) expression profiles with both leading to higher HO-1 levels than heme-Hpx (see Tolosano, E., Fagoonee, S., Morello, N., Vinchi, F. & Fiorito, V. Heme Scavenging and the Other Facets of Hemopexin. Antioxid. Redox Signal. 12, 305-320 (2010)). HSA has also been shown to reduce neural heme toxicity at equimolar concentrations.

Another benefit of HSA is its extensive ligand binding properties (see Fasano, M. et al. The extraordinary ligand binding properties of human serum albumin. IUBMB Life 57, 787-796 (2005)). This allows for a flexible delivery vehicle of drugs for treatment of the desired condition. For example, as the major store of nitric oxide (NO) in vivo, HSA may be used to deliver NO to the vasculature during states of hemolysis, thus preventing hypertension (see Stamler, J. S. et al. Nitric oxide circulates in mammalian plasma primarily as an S-nitroso adduct of serum albumin. Proc. Natl. Acad. Sci. 89, 7674-7677 (1992); Rungatscher, A. et al. S-nitroso human serum albumin attenuates pulmonary hypertension, improves right ventricular-arterial coupling, and reduces oxidative stress in a chronic right ventricle volume overload model. J. Hear. Lung Transplant. 34, 479-488 (2015); and Orie, N. N., Vallance, P., Jones, D. P. & Moore, K. P. S-nitroso-albumin carries a thiol-labile pool of nitric oxide, which causes venodilation in the rat. Am. J. Physiol. Circ. Physiol. 289, H916-H923 (2005)). Nitrite infusions have already been shown to restrict Hb hypertension during hemolysis (see Minneci, P. C. et al. Nitrite reductase activity of hemoglobin as a systemic nitric oxide generator mechanism to detoxify plasma hemoglobin produced during hemolysis. Am. J. Physiol. Circ. Physiol. 295, H743-H754 (2008)). In one strategy, NO delivery would require binding of NO to the free Cys34 of HSA to form S-NO HSA (HSA-SNO) prior to administration of the cocktail, but could serve as a means to increase NO levels in the blood that may have been scavenged due to cell-free Hb. HSA-SNO may also be used in wound healing applications (see Ganzarolli de Oliveira, M. S-Nitrosothiols as Platforms for Topical Nitric Oxide Delivery. Basic Clin. Pharmacol. Toxicol. 119, 49-56 (2016); and LUO, J. & CHEN, A. F. Nitric oxide: a newly discovered function on wound healing. Acta Pharmacol. Sin. 26, 259-264 (2005)). Finally, HSA-SNO can also have application in the treatment of cyanide poisoning (see Leavesley, H. B., Li, L., Mukhopadhyay, S., Borowitz, J. L. & Isom, G. E. Nitrite-Mediated Antagonism of Cyanide Inhibition of Cytochrome c Oxidase in Dopamine Neurons. Toxicol. Sci. 115, 569-576 (2010)).

In some embodiments, recombinant albumins, such as recombinant human serum albumin, can be used.

In some examples, the colloidally stable heme-albumin complex is stable in aqueous solution after 72 hours of storage at 4° C. without loss or precipitation of heme.

In some examples, the colloidally stable heme-albumin complex exhibits a hydrodynamic diameter of from about 10 nm to about 15 nm, as determined by dynamic light scattering (DLS).

### Methods of Making

Also provided are methods for preparing the colloidally stable heme-albumin complexes described herein.

In some examples, the colloidally stable heme-albumin complex can be formed by first complexing the heme with albumin to form a heme-albumin complex, and then conjugating one or more hydrophilic polymer chains (e.g., PEG chains) to the surface of the heme-albumin complex to form a colloidally stable heme-albumin complex. For example, provided herein are methods for forming a colloidally stable heme-albumin complex that comprise contacting albumin with heme under conditions effective to form a heme-albumin complex; conjugating one or more hydrophilic polymers to the heme-albumin complex to form a crude colloidally stable heme-albumin complex; and filtering the crude colloidally stable heme-albumin complex by ultrafiltration against a filtration membrane, thereby forming a retentate fraction comprising the colloidally stable heme-albumin complex and a permeate fraction comprising low molecular weight contaminants.

In some embodiments, the colloidally stable heme-albumin complex can be substantially free of free heme, aggregated heme particulates, unconjugated hydrophilic polymers, and other low molecular weight contaminants. In certain embodiments, the colloidally stable heme-albumin complex can comprise less than 1% by weight (e.g., less than 0.5%, or less than 0.1%) free heme, aggregated heme particulates, unconjugated hydrophilic polymers, and other low molecular weight contaminants having a molecular weight of less than 50 kDa.

In some examples, contacting albumin with heme under conditions effective to form a heme-albumin complex can comprise incubating the albumin with heme at a pH of 8 or more, such as a pH of from 8 to 10. In some examples, the heme and the albumin are present in the contacting step at a molar ratio of heme:albumin of at least 5:1, such as a molar ratio of from 5:1 to 15:1, such as from 5:1 to 12:1, from 5:1 to 10:1, or from 5:1 to 8:1. In some examples, contacting albumin with heme under conditions effective to form a heme-albumin complex can further comprise neutralizing the heme-albumin complex.

In some examples, the filtration membrane can have a filter rated for removing solutes having a molecular weight of from 1 to 50 kDa. In some examples, filtering the crude colloidally stable heme-albumin complex by ultrafiltration can comprise filtering for at least 3 diafiltrations cycles, such as at least 4 diafiltration cycles, at least 5 diafiltration cycles, at least 6 diafiltration cycles, at least 7 diafiltrations cycle, at least 8 diafiltration cycles, at least 9 diafiltrations cycle, or at least 10 diafiltration cycles. In certain embodiments, the ultrafiltration can comprise cross-flow filtration or tangential flow filtration.

In some examples, the one or more hydrophilic polymers can comprise poly(lactide), poly(glycolide), a poly(orthoesters), a poly(caprolactone), polylysine, poly(ethylene imine), poly(acrylic acid), a poly(urethane), a poly(anhydride), a poly(ester), poly(trimethylene carbonate), poly(ethyleneimine), a poly(acrylic acid), a poly(urethane), a poly(beta amino ester), a poly(alkylene oxide) such as polyethylene glycol (PEG), a zwitterionic polymer, a copolymer thereof, or a blend thereof.

In some examples, conjugating the one or more hydrophilic polymers (e.g., one or more polyethylene glycol (PEG) polymers) to the heme-albumin complex to form the crude PEGylated heme-albumin complex can comprise contacting the heme-albumin complex with a derivatized hydrophilic polymer under conditions permitting formation of a covalent bond between the hydrophilic polymer and the heme-albumin complex so as to form the crude colloidally stable heme-albumin complex.

In certain examples, the one or more hydrophilic polymers can comprise polyethylene glycol (PEG). In some of these examples, conjugating the one or more PEG polymers to the heme-albumin complex to form the crude colloidally stable heme-albumin complex comprises contacting the heme-albumin complex with a derivatized PEG polymer under conditions permitting formation of a covalent bond between the PEG polymer and the heme-albumin complex so as to form the crude colloidally stable heme-albumin complex. The derivatized PEG can comprise, for example, succinimidyl-PEG, cyanuric chloride-PEG, or maleimide-PEG. In some of these examples, conjugating the one or more PEG polymers to the heme-albumin complex to form the crude colloidally stable heme-albumin complex further comprises contacting the heme-albumin complex with a thiolation reagent, such as 2-iminothiolane hydrochloride, to introduce thiol moieties that react with the derivatized PEG.

In some examples, the one or more hydrophilic polymers (e.g., PEG) can each have a molecular weight of from 200 Da to 20,000 Da, such as from 1,000 Da to 10,000 Da, or from 3,000 Da to 6,000 Da, or about 5,000 Da.

In some examples, the method can further comprise filtering the heme-albumin complex by ultrafiltration against a filtration membrane prior to conjugating one or more hydrophilic polymers to the heme-albumin complex, thereby forming a retentate fraction comprising the heme-albumin complex and a permeate fraction comprising low molecular weight contaminants. In some examples, following filtration, the heme-albumin complex is substantially free of free heme, aggregated heme particulates, and other low molecular weight contaminants. For example, in some embodiments, following filtration, the heme-albumin complex comprises less than 1% by weight (e.g., less than 0.5% by weight, or less than 0.1% by weight) free heme, aggregated heme particulates, and other low molecular weight contaminants having a molecular weight of less than 50 kDa.

In other examples, the colloidally stable heme-albumin complex can be formed by first conjugating one or more hydrophilic polymer chains to the surface of the albumin to form polymer-modified albumin, and then complexing the heme with the polymer-modified albumin to form a crude colloidally stable heme-albumin complex. For example, also provided are methods for forming a colloidally stable heme-albumin complex that comprise conjugating one or more hydrophilic polymers to albumin to form polymer-modified albumin; contacting the polymer-modified albumin with heme under conditions effective to form a crude colloidally stable heme-albumin complex; and filtering the crude colloidally stable heme-albumin complex by ultrafiltration against a filtration membrane, thereby forming a retentate fraction comprising the colloidally stable heme-albumin complex and a permeate fraction comprising low molecular weight contaminants.

In some embodiments, the colloidally stable heme-albumin complex can be substantially free of free heme, aggregated heme particulates, unconjugated hydrophilic polymers, and other low molecular weight contaminants. In certain embodiments, the colloidally stable heme-albumin complex can comprise less than 1% by weight (e.g., less than 0.5%, or less than 0.1%) free heme, aggregated heme particulates, unconjugated hydrophilic polymers, and other low molecular weight contaminants having a molecular weight of less than 50 kDa.

In some examples, contacting the polymer-modified albumin with heme under conditions effective to form a crude colloidally stable heme-albumin complex can comprise incubating the polymer-modified albumin with heme at a pH of 8 or more, such as a pH of from 8 to 10. In some examples, the heme and the polymer-modified albumin are present in the contacting step at a molar ratio of heme:polymer-modified albumin of at least 5:1, such as a molar ratio of from 5:1 to 15:1, such as from 5:1 to 12:1, from 5:1 to 10:1, or from 5:1 to 8:1. In some examples, contacting polymer-modified albumin with heme under conditions effective to form a crude colloidally stable heme-albumin complex further comprises neutralizing the crude colloidally stable heme-albumin complex.

In some examples, the filtration membrane can have a filter rated for removing solutes having a molecular weight of from 1 to 50 kDa. In some examples, filtering the crude colloidally stable heme-albumin complex by ultrafiltration can comprise filtering for at least 3 diafiltrations cycles, such as at least 4 diafiltration cycles, at least 5 diafiltration cycles, at least 6 diafiltration cycles, at least 7 diafiltrations cycle, at least 8 diafiltration cycles, at least 9 diafiltrations cycle, or at least 10 diafiltration cycles. In certain embodiments, the ultrafiltration can comprise cross-flow filtration or tangential flow filtration.

In some examples, the one or more hydrophilic polymers can comprise poly(lactide), poly(glycolide), a poly(orthoesters), a poly(caprolactone), polylysine, poly(ethylene imine), poly(acrylic acid), a poly(urethane), a poly(anhydride), a poly(ester), poly(trimethylene carbonate), poly(ethyleneimine), a poly(acrylic acid), a poly(urethane), a poly(beta amino ester), a poly(alkylene oxide) such as polyethylene glycol (PEG), a zwitterionic polymer, a copolymer thereof, or a blend thereof.

In some examples, conjugating the one or more hydrophilic polymers (e.g., one or more polyethylene glycol (PEG) polymers) to the albumin to form the polymer-modified albumin can comprise contacting the albumin with a derivatized hydrophilic polymer under conditions permitting formation of a covalent bond between the hydrophilic polymer and the albumin so as to form the polymer-modified albumin.

In certain examples, the one or more hydrophilic polymers can comprise polyethylene glycol (PEG). In some of these examples, conjugating the one or more PEG polymers to the albumin to form PEGylated albumin comprises contacting the albumin with a derivatized PEG polymer under conditions permitting formation of a covalent bond between the PEG polymer and the albumin so as to form the PEGylated albumin. The derivatized PEG can comprise, for example, succinimidyl-PEG, cyanuric chloride-PEG, or maleimide-PEG. In some of these examples, conjugating the one or more PEG polymers to the albumin to form the PEGylated albumin further comprises contacting the albumin with a thiolation reagent, such as 2-iminothiolane hydrochloride, to introduce thiol moieties that react with the derivatized PEG.

In some examples, the one or more hydrophilic polymers (e.g., PEG) can each have a molecular weight of from 200 Da to 20,000 Da, such as from 1,000 Da to 10,000 Da, or from 3,000 Da to 6,000 Da, or about 5,000 Da.

In some examples, the method further comprises filtering the polymer-modified albumin by ultrafiltration against a filtration membrane prior to contacting the polymer-modified albumin with heme under conditions effective to form a crude colloidally stable heme-albumin complex, thereby forming a retentate fraction comprising the polymer-modified albumin and a permeate fraction comprising low molecular weight contaminants. In some examples, following filtration, the polymer-modified albumin is substantially free of unconjugated hydrophilic polymers and other low molecular weight contaminants. For example, following filtration, the polymer-modified albumin comprises less than 1% by weight (e.g., less than 0.5% by weight, or less than 0.1% by weight) free unconjugated hydrophilic polymers and other low molecular weight contaminants having a molecular weight of less than 50 kDa.

### Compositions

Provided herein are compositions that comprise the colloidally stable heme-albumin complexes described herein dissolved or dispersed in an aqueous carrier. In some embodiments, the compositions can comprise a therapeutically effective amount of the colloidally stable heme-albumin complexes dissolved or dispersed in an aqueous carrier.

In some examples, the composition can comprise a therapeutically effective amount of the colloidally stable heme-albumin complexes to reducing inflammation in a subject in need thereof.

In some examples, the composition can comprise a therapeutically effective amount of the colloidally stable heme-albumin complexes to treat or prevent an ophthalmological disorder in a subject in need thereof.

In some embodiments, the composition can further comprise an additional active agent (in addition to the colloidally stable heme-albumin complexes). The additional active agent can comprise a therapeutic, diagnostic, and/or prophylactic agent. The active agent can be a small molecule active agent and/or a biomolecule, such as an enzyme, protein, growth factor, polypeptide, polysaccharide, lipid, or nucleic acid. Suitable small molecule active agents include organic and organometallic compounds. In some instances, the small molecule active agent has a molecular weight of less than about 2000 g/mol, preferably less than about 1500 g/mol, more preferably less than about 1200 g/mol, most preferably less than about 1000 g/mol. In other embodiments, the small molecule active agent has a molecular weight less than about 500 g/mol. The small molecule active agent can be a hydrophilic, hydrophobic, or amphiphilic compound. Biomolecules typically have a molecular weight of greater than about 2000 g/mol and may be composed of repeat units such as amino acids (peptide, proteins, enzymes, etc.) or nitrogenous base units (nucleic acids).

In certain embodiments, the additional active agent is an ophthalmic drug. In particular embodiments, the additional active agent is a drug used to treat, prevent or diagnose a disease or disorder of the posterior segment eye. Non-limiting examples of ophthalmic drugs include anti-glaucoma agents, anti-angiogenesis agents, anti-infective agents, anti-inflammatory agents, growth factors, immunosuppressant agents, anti-allergic agents, and combinations thereof.

Representative anti-glaucoma agents include prostaglandin analogs (such as travoprost, bimatoprost, and latanoprost), beta-andrenergic receptor antagonists (such as timolol, betaxolol, levobetaxolol, and carteolol), alpha-2 adrenergic receptor agonists (such as brimonidine and apraclonidine), carbonic anhydrase inhibitors (such as brinzolamide, acetazolamine, and dorzolamide), miotics (i.e., parasympathomimetics, such as pilocarpine and ecothiopate), seretonergics muscarinics, dopaminergic agonists, and adrenergic agonists (such as apraclonidine and brimonidine).

Representative anti-angiogenesis agents include, but are not limited to, antibodies to vascular endothelial growth factor (VEGF) such as bevacizumab (AVASTIN®) and rhuFAb V2 (ranibizumab, LUCENTIS@), and other anti-VEGF compounds such as aflibercept (EYLEA®), faricimab, and brolucizumab; MACUGEN@(pegaptanim sodium, anti-VEGF aptamer or EYE001) (Eyetech Pharmaceuticals); pigment epithelium derived factor(s) (PEDF); COX-2 inhibitors such as celecoxib (CELEBREX®) and rofecoxib (VIOXX@); interferon alpha; interleukin-12 (IL-12); thalidomide (THALOMID®) and derivatives thereof such as lenalidomide (REVLIMID®); squalamine; endostatin; angiostatin; ribozyme inhibitors such as ANGIOZYME® (Sirna Therapeutics); multifunctional antiangiogenic agents such as NEOVASTAT@(AE-941) (Aeterna Laboratories, Quebec City, Canada); receptor tyrosine kinase (RTK) inhibitors such as sunitinib (SUTENT®); tyrosine kinase inhibitors such as sorafenib (Nexavarg) and erlotinib (Tarceva®); antibodies to the epidermal grown factor receptor such as panitumumab (VECTIBIX®) and cetuximab (ERBITUX®); as well as other anti-angiogenesis agents known in the art.

Anti-infective agents include antiviral agents, antibacterial agents, antiparasitic agents, and anti-fungal agents. Representative antiviral agents include ganciclovir and acyclovir. Representative antibiotic agents include aminoglycosides such as streptomycin, amikacin, gentamicin, and tobramycin, ansamycins such as geldanamycin and herbimycin, carbacephems, carbapenems, cephalosporins, glycopeptides such as vancomycin, teicoplanin, and telavancin, lincosamides, lipopeptides such as daptomycin, macrolides such as azithromycin, clarithromycin, dirithromycin, and erythromycin, monobactams, nitrofurans, penicillins, polypeptides such as bacitracin, colistin and polymyxin B, quinolones, sulfonamides, polyhexamethylene biguanide (PHMB), and tetracyclines.

In some cases, the active agent is an anti-allergic agent such as olopatadine and epinastine.

Anti-inflammatory agents include both non-steroidal and steroidal anti-inflammatory agents. Suitable steroidal active agents include glucocorticoids, progestins, mineralocorticoids, and corticosteroids.

The ophthalmic drug may be present in its neutral form, or in the form of a pharmaceutically acceptable salt. In some cases, it may be desirable to prepare a formulation containing a salt of an active agent due to one or more of the salt's advantageous physical properties, such as enhanced stability or a desirable solubility or dissolution profile.

Generally, pharmaceutically acceptable salts can be prepared by reaction of the free acid or base forms of an active agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an active agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, Md., 2000, p. 704. Examples of ophthalmic drugs sometimes administered in the form of a pharmaceutically acceptable salt include timolol maleate, brimonidine tartrate, and sodium diclofenac.

In some cases, the active agent is a diagnostic agent imaging or otherwise assessing the eye. Exemplary diagnostic agents include paramagnetic molecules, fluorescent compounds, magnetic molecules, and radionuclides, x-ray imaging agents, and contrast media.

The compositions described herein may be prepared using a physiological saline solution as a vehicle. The pH of the composition may be maintained at a substantially neutral pH (for example, about 7.4, in the range of about 6.5 to about 7.4, etc.) with an appropriate buffer system as known to one skilled in the art (for example, acetate buffers, citrate buffers, phosphate buffers, borate buffers).

Any diluent used in the preparation of the compositions may preferably be selected so as not to unduly affect the biological activity of the composition. Examples of such diluents which are especially for injectable ophthalmic compositions are water, various saline, organic, or inorganic salt solutions, Ringer's solution, dextrose solution, and Hank's solution.

In addition, the compositions may include additives such other buffers, diluents, carriers, adjuvants, or excipients. Any pharmaceutically acceptable buffer suitable for application to the eye may be used, e.g., tris or phosphate buffers. Other agent may be employed in the formulation for a variety of purposes. For example, buffering agents, preservatives, co-solvents, surfactants, oils, humectants, emollients, chelating agents, stabilizers or antioxidants may be employed. Water soluble preservatives which may be employed include, but are not limited to, benzalkonium chloride, chlorobutanol, thimerosal, sodium bisulfate, phenylmercuric acetate, phenylmercuric nitrate, ethyl alcohol, methylparaben, polyvinyl alcohol, benzyl alcohol and phenylethyl alcohol. A surfactant may be Tween 80.

Other vehicles that may be used include, but are not limited to, polyvinyl alcohol, povidone, hydroxypropyl methyl cellulose, poloxamers, carboxymethyl cellulose, hydroxyethyl cellulose, or purified water. Tonicity adjustors may be included, for example, sodium chloride, potassium chloride, mannitol, or glycerin. Antioxidants include, but are not limited to, sodium metabisulfite, sodium thiosulfate, acetylcysteine, butylated hydroxyanisole, or butylated hydroxytoluene. The indications, effective doses, formulations, contraindications, etc. of the components in the ophthalmic composition are available and are known to one skilled in the art.

These agents may be present in individual amounts from about 0.001% to about 5% by weight and preferably about 0.01% to about 2% by weight in the formulation. Suitable water soluble buffering agents that may be employed are sodium carbonate, sodium borate, sodium phosphate, sodium acetate, or sodium bicarbonate, as approved by the U.S. FDA for the desired route of administration. These agents may be present in amounts sufficient to maintain a pH of the system between about 2 to about 9 and preferably about 4 to about 8. As such, the buffering agent may be as much as about 5% (w/w) of the total ocular therapeutic composition. Electrolytes such as, but limited to, sodium chloride and potassium chloride may be also included in the formulation.

In some embodiments, the composition further comprises a hydrogel. In some embodiments, the hydrogel comprises a polymer composition, for example a homopolymer, a copolymer, or combinations thereof. In some embodiments, the hydrogel comprises one or more hydrophilic polymers, i.e., a polymer having at least 0.1 wt. % solubility in water, for example having at least 0.5 wt. % solubility. In some embodiments, the hydrophilic polymer has a solubility of at least 1 mg/mL.

In some embodiments, the polymer composition may comprise one or more vinyl alcohol residues. In some embodiments, the polymer composition may comprise one or more acrylamide residues. In some embodiments, the polymer composition may comprise one or more residues selected from a polyethylene glycol derivative or a functionalized polyethylene glycol. In some embodiments, the polymer composition may comprise one or more acrylate residues or one or more methacrylate residues. In some embodiments, the polymer composition may comprise one or more residues selected from acrylamide, N-omithine acrylamide, N-(2-hydroxypropyl)acrylamide, hydroxyethylacrylate, hydroxyethylmethacrylate, polyethyleneglycol acrylates, polyethylenegylcol methacrylates, N-vinylpyrrolidinone, N-phenylacrylamide, dimethylaminopropyl methacrylamide, acrylic acid, benzylmethacrylamide, methylthioethylacrulamide, or combinations thereof.

Representative examples of hydrogels which can be used include, but are not limited to, hyaluronic acid, collagen, gellan, silk, fibrin, alginate, chitosan, polyacrylamides and methacrylate derivatives thereof, polyacrylic acid and methacrylate derivatives thereof, polyvinyl alcohol, polyethylene glycol and derivatives thereof, polypropylene glycol and derivatives thereof, or combinations thereof.

In some embodiments, the hydrogel comprises a hyaluronate derivative, for example poly(N-isopropylacrylamide) grafted sodium hyaluronate.

### Methods of Use

The term “administering” or “administration” of a disclosed therapeutic composition to a subject includes any route of introducing or delivering to a subject the device to perform its intended function. Administration can be carried out by any suitable route, including orally, intranasally, parenterally (intravenously, intramuscularly, intraperitoneally, or subcutaneously), or topically. Administration includes self-administration and the administration by another. In some instances, administration is via injection to the eye, including intraocular injection.

It is also to be appreciated that the various modes of treatment or prevention of medical diseases and conditions as described are intended to mean “substantial,” which includes total but also less than total treatment or prevention, and wherein some biologically or medically relevant result is achieved. The treatment may be a continuous prolonged treatment for a chronic disease or a single, or few time administrations for the treatment of an acute condition.

The term “separate” administration refers to an administration of at least two active ingredients at the same time or substantially the same time by different routes.

The term “sequential” administration refers to administration of at least two active ingredients at different times, the administration route being identical or different. More particularly, sequential use refers to the whole administration of one of the active ingredients before administration of the other or others commences. It is thus possible to administer one of the active ingredients over several minutes, hours, or days before administering the other active ingredient or ingredients. The term “sequential” therefore is different than “simultaneous” administration.

The term “simultaneous” administration refers to the administration of at least two active ingredients by the same route at the same time or at substantially the same time.

The term “therapeutic” as used herein means a treatment and/or prophylaxis. A therapeutic effect is obtained by suppression, remission, or eradication of a disease state.

In some embodiments, the compositions described herein can be administered to a subject to deliver the colloidally stable heme-albumin complexes described herein for therapeutic benefit.

In some embodiments, the composition can be administered to a subject to reduce inflammation in the subject.

The present disclosure further provides methods of treating an ophthalmological disease or disorder by administering a therapeutically effective amount of the compositions described herein. In some embodiments, the disclosed methods pertain to treatment of an ophthalmological disorder comprising injecting a therapeutically effective amount of a composition comprising colloidally stable heme-albumin complex into the eye of a subject. The subject can be a patient; and the patient can have been diagnosed with an ophthalmological disorder. In some aspects, the method can further comprise diagnosing a subject with an ophthalmological disorder.

Also provided are methods of treating an ophthalmological disorder in a subject in need thereof comprising injecting into the eye of the subject a therapeutically effective amount of a composition comprising colloidally stable heme-albumin complex, as described herein.

The ophthalmological disorder can be acute macular neuroretinopathy; Behcet's disease; neovascularization, including choroidal neovascularization; diabetic uveitis; histoplasmosis; infections, such as fungal or viral-caused infections; macular degeneration, such as acute macular degeneration (AMD), including wet AMD, non-exudative AMD and exudative AMD; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic ophthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, a cancer, and glaucoma. In certain instances, the ophthalmological disorder is wet age-related macular degeneration (wet AMD), a cancer, neovascularization, macular edema, or edema.

In certain embodiments, the ophthalmological disorder can comprise an inflammation-mediated disorder. “Inflammation-mediated” in relation to an ocular condition means any condition of the eye which can benefit from treatment with an anti-inflammatory agent, and is meant to include, but is not limited to, uveitis, macular edema, acute macular degeneration, retinal detachment, ocular tumors, fungal or viral infections, multifocal choroiditis, diabetic retinopathy, uveitis, proliferative vitreoretinopathy (PVR), sympathetic ophthalmia, Vogt-Koyanagi-Harada (VKH) syndrome, histoplasmosis, and uveal diffusion

In a further particular aspect, the ophthalmological disorder is wet age-related macular degeneration (wet AMD). In a further particular aspect, the ophthalmological disorder is dry age-related macular degeneration (dry AMD).

In a further aspect, the ophthalmological disorder can comprise a retinal degenerative disease, such as PCR or geographic atrophy.

In various aspects, the injection for treatment of an ophthalmological disorder can be injection to the vitreous chamber of the eye. In some cases, the injection is an intravitreal injection, a subconjunctival injection, a subtenon injection, a retrobulbar injection, or a suprachoroidal injection.

“Ocular region” or “ocular site” means any area of the ocular globe (eyeball), including the anterior and posterior segment of the eye, and which generally includes, but is not limited to, any functional (e.g., for vision) or structural tissues found in the eyeball, or tissues or cellular layers that partly or completely line the interior or exterior of the eyeball. Specific examples of areas of the eyeball in an ocular region include, but are not limited to, the anterior chamber, the posterior chamber, the vitreous cavity, the choroid, the suprachoroidal space, the conjunctiva, the subconjunctival space, the episcleral space, the intracorneal space, the subretinal space, sub-Tenon's space, the epicorneal space, the sclera, the pars plana, surgically-induced avascular regions, the macula, and the retina.

“Ophthalmological disorder” can mean a disease, ailment or condition which affects or involves the eye or one of the parts or regions of the eye. Broadly speaking, the eye includes the eyeball, including the cornea, and other tissues and fluids which constitute the eyeball, the periocular muscles (such as the oblique and rectus muscles) and the portion of the optic nerve which is within or adjacent to the eyeball.

“Glaucoma” means primary, secondary and/or congenital glaucoma. Primary glaucoma can include open angle and closed angle glaucoma. Secondary glaucoma can occur as a complication of a variety of other conditions, such as injury, inflammation, pigment dispersion, vascular disease and diabetes. The increased pressure of glaucoma causes blindness because it damages the optic nerve where it enters the eye. Thus, in one nonlimiting embodiment, by lowering reactive oxygen species, STC-1, or MSCs which express increased amounts of STC-1, may be employed in the treatment of glaucoma and prevent or delay the onset of blindness.

“Injury” or “damage” in relation to an ocular condition are interchangeable and refer to the cellular and morphological manifestations and symptoms resulting from an inflammatory-mediated condition, such as, for example, inflammation, as well as tissue injuries caused by means other than inflammation, such as chemical injury, including chemical burns, as well as injuries caused by infections, including but not limited to, bacterial, viral, or fungal infections.

“Intraocular” means within or under an ocular tissue. An intraocular administration of an ocular therapeutic composition includes administration of the ocular therapeutic composition to a sub-tenon, subconjunctival, suprachoroidal, subretinal, intravitreal, anterior chamber, and the like location. An intraocular administration of an ocular therapeutic composition excludes administration of the drug delivery system to a topical, systemic, intramuscular, subcutaneous, intraperitoneal, and the like location.

“Macular degeneration” refers to any of a number of disorders and conditions in which the macula degenerates or loses functional activity. The degeneration or loss of functional activity can arise as a result of, for example, cell death, decreased cell proliferation, loss of normal biological function, or a combination of the foregoing. Macular degeneration can lead to and/or manifest as alterations in the structural integrity of the cells and/or extracellular matrix of the macula, alteration in normal cellular and/or extracellular matrix architecture, and/or the loss of function of macular cells. The cells can be any cell type normally present in or near the macula including RPE cells, photoreceptors, and capillary endothelial cells. Age-related macular degeneration, or ARMD, is the major macular degeneration related condition, but a number of others are known including, but not limited to, Best macular dystrophy, Stargardt macular dystrophy, Sorsby fundus dystrophy, Mallatia Leventinese, Doyne honeycomb retinal dystrophy, and RPE pattern dystrophies. Age-related macular degeneration (AMD) is described as either “dry” or “wet.” The wet, exudative, neovascular form of AMD affects about 10-20% of those with AMD and is characterized by abnormal blood vessels growing under or through the retinal pigment epithelium (RPE), resulting in hemorrhage, exudation, scarring, or serous retinal detachment. Eighty to ninety percent of AMD patients have the dry form characterized by atrophy of the retinal pigment epithelium and loss of macular photoreceptors. Drusen may or may not be present in the macula. There may also be geographic atrophy of retinal pigment epithelium in the macula accounting for vision loss. At present there is no cure for any form of AMD, although some success in attenuation of wet AMD has been obtained with photodynamic and especially anti-VEGF therapy.

“Drusen” is debris-like material that accumulates with age below the RPE. Drusen is observed using a funduscopic eye examination. Normal eyes may have maculas free of drusen, yet drusen may be abundant in the retinal periphery. The presence of soft drusen in the macula, in the absence of any loss of macular vision, is considered an early stage of AMD. Drusen contains a variety of lipids, polysaccharides, and glycosaminoglycans along with several proteins, modified proteins or protein adducts. There is no generally accepted therapeutic method that addresses drusen formation and thereby manages the progressive nature of AMD.

“Ocular neovascularization” (ONV) is used herein to refer to choroidal neovascularization or retinal neovascularization, or both.

“Retinal neovascularization” (RNV) refers to the abnormal development, proliferation, and/or growth of retinal blood vessels, e.g., on the retinal surface.

“Subretinal neovascularization” (SRNVM) refers to the abnormal development, proliferation, and/or growth of blood vessels beneath the surface of the retina.

“Cornea” refers to the transparent structure forming the anterior part of the fibrous tunic of the eye. It consists of five layers, specifically: 1) anterior corneal epithelium, continuous with the conjunctiva; 2) anterior limiting layer (Bowman's layer); 3) substantia propria, or stromal layer; 4) posterior limiting layer (Descemet's membrane); and 5) endothelium of the anterior chamber or keratoderma.

“Retina” refers to the innermost layer of the ocular globe surrounding the vitreous body and continuous posteriorly with the optic nerve. The retina is composed of layers including the: 1) internal limiting membrane; 2) nerve fiber layer; 3) layer of ganglion cells; 4) inner plexiform layer; 5) inner nuclear layer; 6) outer plexiform layer; 7) outer nuclear layer; 8) external limiting membrane; and 9) a layer of rods and cones.

“Retinal degeneration” refers to any hereditary or acquired degeneration of the retina and/or retinal pigment epithelium. Non-limiting examples include retinitis pigmentosa, Best's Disease, RPE pattern dystrophies, and age-related macular degeneration.

In various aspects, a method of treating an ophthalmological disorder may comprise treatment of various ocular diseases or conditions of the retina, including the following: maculopathies/retinal degeneration: macular degeneration, including age-related macular degeneration (ARMD), such as non-exudative age-related macular degeneration and exudative age-related macular degeneration; choroidal neovascularization; retinopathy, including diabetic retinopathy, acute and chronic macular neuroretinopathy, central serous chorioretinopathy; and macular edema, including cystoid macular edema, and diabetic macular edema. Uveitis/retinitis/choroiditis: acute multifocal placoid pigment epitheliopathy, Behcet's disease, birdshot retinochoroidopathy, infectious (syphilis, Lyme Disease, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, and Vogt-Koyanagi-Harada syndrome. Vascular diseases/exudative diseases: retinal arterial occlusive disease, central retinal vein occlusion, disseminated intravascular coagulopathy, branch retinal vein occlusion, hypertensive fundus changes, ocular ischemic syndrome, retinal arterial microaneurysms, Coats disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, Eales disease, Traumatic/surgical diseases: sympathetic ophthalmia, uveitic retinal disease, retinal detachment, trauma, laser, PDT, photocoagulation, hypoperfusion during surgery, radiation retinopathy, bone marrow transplant retinopathy. Proliferative disorders: proliferative vitreal retinopathy and epiretinal membranes, proliferative diabetic retinopathy. Infectious disorders: ocular histoplasmosis, ocular toxocariasis, ocular histoplasmosis syndrome (OHS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis. Genetic disorders: retinitis pigmentosa, systemic disorders with associated retinal dystrophies, congenital stationary night blindness, cone dystrophies, Stargardt's disease and fundus flavimaculatus, Best's disease, pattern dystrophy of the retinal pigment epithelium, X-linked retinoschisis, Sorsby's fundus dystrophy, benign concentric maculopathy, Bietti's crystalline dystrophy, pseudoxanthoma elasticum. Retinal tears/holes: retinal detachment, macular hole, giant retinal tear. Tumors: retinal disease associated with tumors, congenital hypertrophy of the RPE, posterior uveal melanoma, choroidal hemangioma, choroidal osteoma, choroidal metastasis, combined hamartoma of the retina and retinal pigment epithelium, retinoblastoma, vasoproliferative tumors of the ocular fundus, retinal astrocytoma, and intraocular lymphoid tumors. Miscellaneous: punctate inner choroidopathy, acute posterior multifocal placoid pigment epitheliopathy, myopic retinal degeneration, acute retinal pigment epithelitis and the like.

An anterior ocular condition is a disease, ailment or condition which affects or which involves an anterior (i.e., front of the eye) ocular region or site, such as a periocular muscle, an eyelid or an eyeball tissue or fluid which is located anterior to the posterior wall of the lens capsule or ciliary muscles. Thus, an anterior ocular condition primarily affects or involves the conjunctiva, the cornea, the anterior chamber, the iris, the posterior chamber (behind the iris but in front of the posterior wall of the lens capsule), the lens or the lens capsule and blood vessels and nerve which vascularize or innervate an anterior ocular region or site.

Thus, an anterior ocular condition can include a disease, ailment or condition, such as for example, aphakia; pseudophakia; astigmatism; blepharospasm; cataract; posterior capsule opacification (PCO); conjunctival diseases; conjunctivitis, including, but not limited to, atopic keratoconjunctivitis; corneal injuries, including, but not limited to, injury to the corneal stromal areas; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered an anterior ocular condition because a clinical goal of glaucoma treatment can be to reduce a hypertension of aqueous fluid in the anterior chamber of the eye (i.e., reduce intraocular pressure).

Other diseases or disorders of the eye which may be treated in accordance with the present invention include, but are not limited to, ocular cicatricial pemphigoid (OCP), Stevens Johnson syndrome and cataracts.

A posterior ocular condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e., the optic disc), and blood vessels and nerves which vascularize or innervate a posterior ocular region or site. Thus, a posterior ocular condition can include a disease, ailment or condition, such as for example, acute macular neuroretinopathy; Behcet's disease; choroidal neovascularization; diabetic retinopathy; uveitis; ocular histoplasmosis; infections, such as fungal or viral-caused infections; macular degeneration, such as acute macular degeneration, non-exudative age-related macular degeneration and exudative age-related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial or venous occlusive disease, retinal detachment, uveitic retinal disease; sympathetic ophthalmia; Vogt-Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal ganglion cells or retinal nerve fibers (i.e., neuroprotection).

In some embodiments, the ophthalmic disorder is ocular inflammation resulting from, e.g., iritis, conjunctivitis, seasonal allergic conjunctivitis, acute and chronic endophthalmitis, anterior uveitis, uveitis associated with systemic diseases, posterior segment uveitis, chorioretinitis, pars planitis, masquerade syndromes including ocular lymphoma, pemphigoid, scleritis, keratitis, severe ocular allergy, corneal abrasion and blood-aqueous barrier disruption. In yet another embodiment, the ophthalmic disorder is post-operative ocular inflammation resulting from, for example, photorefractive keratectomy, cataract removal surgery, intraocular lens implantation, vitrectomy, corneal transplantation, forms of lamellar keratectomy (DSEK, etc.), and radial keratotomy.

In various aspects, the injection for treatment of an ophthalmological disorder can be injection to the vitreous chamber of the eye. In some cases, the injection is an intravitreal injection, a subconjunctival injection, a subtenon injection, a retrobulbar injection, or a suprachoroidal injection.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example 1: Scalable Manufacturing Platform for the Production of PEGylated Heme Albumin

Cell-free heme, which was previously shown to have adverse effects on the innate immune system, does not induce inflammation when bound to a protein carrier via over-expression of the enzyme heme-oxygenase 1 (HO-1). Studies in mouse macrophage cell culture and human endothelial cells have confirmed HO-1 catalyzed breakdown of protein bound heme into biliverdin, iron, and CO, which elicits anti-inflammatory effects. However, in order to fully realize the anti-inflammatory therapeutic effects of heme, a colloidally stable heme protein carrier must be developed.

To accomplish this goal, we incorporated multiple heme molecules into human serum albumin (HSA) via partial unfolding of HSA at basic pH followed by refolding at neutral pH, and subsequently conjugated the surface of the heme-HSA complex with polyethylene glycol to stabilize heme-HSA. Quantification studies confirmed that a maximum of 5-6 hemes could be bound to HSA without precipitation or degradation of heme-HSA. DLS, SEC-HPLC, and MADI-TOF MS confirmed the increase in hydrodynamic diameter and molecular weight, respectively, upon PEGylation of heme-HSA. Furthermore, PEG-heme-HSA was stable upon exposure to different pH environments, freeze-thaw cycles, and storage at 4° C. Taken together, this example describes a synthesis and purification platform for the production of PEGylated heme-incorporated HSA.

### Materials and Methods

Materials. 25 wt % human serum albumin solution manufactured by Octapharma® was acquired from Nova Biologics (Oceanside, CA). PEG-maleimide (5000 Da) was purchased from Laysan Bio (Arab, AL). Hemin, 5,5-dithio-bis-(2-nitrobenzoic acid) (DTNB), sodium phosphate dibasic (Na₂HPO₄), sodium phosphate monobasic (NaH₂PO₄), sodium chloride (NaCl), potassium chloride (KCl), sodium hydroxide (NaOH), and tris-HCl, were purchased from Sigma-Aldrich (St. Louis, MO). Tangential flow filtration (TFF) modules with a 50 kDa molecular weight cut-off (MWCO) were purchased from Repligen Co. (Waltham, MA). All other chemicals and supplies were purchased from Fisher Scientific (Pittsburgh, PA).

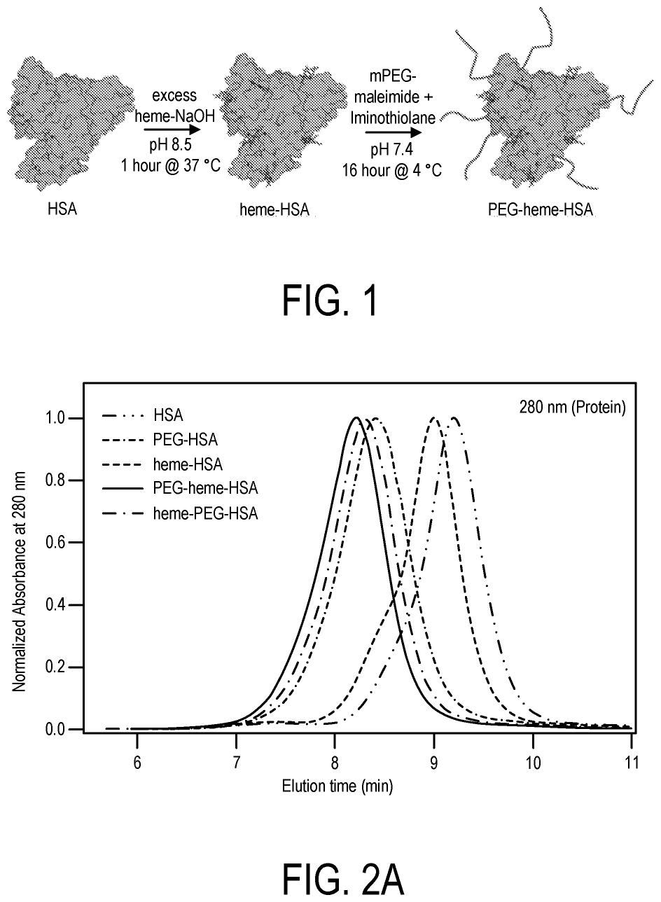

Preparation of Heme-HSA. 10 mM hemin stock solution was prepared by dissolving heme in 0.1 M NaOH. 10 mL of 10 mM hemin stock solution was mixed with 15 mL of HSA stock solution at 100 mg/mL and incubated at 37° C. for 1 hour. The pH of the resulting solution was adjusted to 7.4 using phosphoric acid. The heme-HSA solution was sterile filtered using 0.2 um filters to remove any large aggregates, and subjected to 10 diafiltration cycles via TFF over a 50 kDa MWCO HF module using PBS as the exchange/wash buffer. The preparation of heme-HSA is shown in FIG. 1.

PEGylation Reaction. PEGylation of HSA was performed before and after incorporation of heme using thiol-maleimide chemistry. For the product heme-PEG-HSA, HSA was incubated with 2× molar excess of iminothiolane hydrochloride (Fisher Scientific) and 10× excess mPEG-maleimide (5000 Da) (Laysan Bio) at 4° C. for 16 hours. Unreacted components were removed via diafiltration using TFF HF modules with 50 kDa MWCO in PBS buffer. 4 mM hemin solution in 0.1 M NaOH was then added to PEG-HSA and incubated for 1 hour at 37° C. TFF was performed again to remove excess reagents. For the product PEG-heme-HSA, heme-HSA was prepared as described previously. PEGylation was carried out by incubating heme-HSA with 2× excess iminothiolane and 10× excess mPEG-maleimide (5000 Da). TFF was performed to remove unreacted components. All final materials were filtered through 0.2 μm syringe filters and stored at −80° C. The preparation of PEG-heme-HSA is shown in FIG. 1.

Total Protein Measurement. The concentration of HSA was determined by UV-visible spectrometry using an extinction coefficient of 35,700 M⁻¹ cm⁻¹ at 280 nm. Spectrophotometric absorbance measurements were obtained using a HP 8452A diode array spectrophotometer (Olis, Bogart, GA).

Thiol Quantification Assay. A stock solution of 5,5-dithio-bis-(2-nitrobenzoic acid) (DTNB) was diluted 50 times in reaction buffer (0.1 M sodium phosphate, 1 mM EDTA pH 8.0) to yield a 0.2 mM working solution of DTNB. Protein samples were diluted to 1-4 mg/mL in reaction buffer. 50 μL of protein sample was added to 150 μL of DTNB working solution and allowed to react for 2 mins. Absorbance readings were taken in a micro well plate reader at 412 nm. The readings were adjusted for the initial absorbance, and the concentration of thiols in the sample was determined using an extinction coefficient of 14,100 M¹ cm⁻¹ at 412 nm.

Heme Incorporation Quantification. Heme-HSA was synthesized as previously described at varying heme to HSA molar ratios—2×, 6×, 10×, and 12′. Samples of HSA were collected at multiple points in the synthesis process—pre-reaction HSA pH 7.0, heme-NaOH+HSA start of reaction pH 8.5, heme-NaOH+HSA end of reaction pH 8.5, heme-NaOH+HSA pH 7.4, and final product heme-HSA pH 7.4 after TFF diafiltration. All final products were centrifuged at 20,000×g for 3 mins to precipitate unbound heme. The supernatant was removed, and the heme pellet was solubilized in 1 mL of 0.1 M NaOH. The concentration of unbound heme (i.e. from NaOH solubilized heme pellet) in each sample was determined using the absorbance at the 398 nm peak, and using an extinction coefficient of 12 x10⁴ M⁻¹ cm⁻¹. The difference in the mass of free heme solubilized in NaOH before reaction (total heme), and after centrifugation to form the heme pellet (unbound heme) was used to determine the amount of bound heme in the heme-HSA sample.

Quaternary Structure. Samples were separated on an analytical Acclaim SEC-1000 (4.6-300 mm) column (Thermo Fisher Scientific, Waltham, MA) attached to a Dionex UltiMate 3000 UHPLC/HPLC system (Thermo Fisher Scientific, Waltham, MA). The mobile phase consisted of 50 mM sodium phosphate buffer (PB) at pH 7.4. Chromeleon 7 software was used to control HPLC parameters such as flow rate (0.35 mL/min), UV-visible absorbance detection (280 nm and 398 nm), and fluorescence detection (excitation at 285 nm, emission at 330 nm). All samples were filtered through 0.2 μm syringe filters before size exclusion chromatography HPLC (SEC-HPLC) analysis.

Hydrodynamic Diameter. The hydrodynamic diameter of protein samples was measured using a BI-200SM goniometer (Brookhaven Instruments Corp., Holtsville, NY) at an angle of 90° and wavelength of 637 nm. Protein samples were diluted to ˜0.5-1 mg/mL concentration in deionized water. The hydrodynamic diameter was obtained by using average values from the non-linear least squared (NNLS) algorithm in the instrument software.

Secondary Structure. The far UV circular dichroism (CD) spectra of protein samples was measured using a JASCO J-815 CD (JASCO, Easton, MD) spectrometer. Samples were diluted to 10 μM (protein basis) in deionized water and the absorbance was measured from 200 to 260 nm. For each measurement, spectra were obtained by averaging 3 repeated scans. Baseline subtraction was performed, and the absorbance was normalized using the protein concentration. The molar ellipticity was reported in units of deg cm² dmol⁻¹. The α-helix peak at 222 nm was used to determine the change in α-helical content of samples.

Mass Spectral Analysis. Samples were diluted to 0.5 mg/mL on a protein basis in deionized water. A saturated solution of α-cyano-4-hydroxycinnamic acid (CHCA) matrix was prepared in 50% v/v acetonitrile with 0.1% trifluoroacetic acid (TFA). 1 μL of the mixture of the matrix and protein solution was deposited on a matrix assisted laser desorption/ionization (MALDI) plate and run on a Microflex MALDI-TOF (time of flight) MS (mass spectrometry) system (Bruker, Billerica, MA). The data was analyzed using Flex Analysis software (Bruker, Billerica, MA).

Stability Analysis. Heme-HSA and PEG-heme-HSA samples were either stored at 4° C., or −80° C. Frozen samples (−80° C.) were thawed at room temperature and stored at 4° C. for 0 hour, 24 hours, and 72 hours to test for structural changes caused by the freeze-thaw cycle and extended storage at 4° C. All samples were subjected to SEC-HPLC analysis as previously described. Elution times of the peaks and respective areas under the curve were quantified to determine the structural stability of the products when stored at room temperature and after exposure to a freeze-thaw cycle.

Statistical Analysis. In this Example, all statistical analysis was performed using ANOVA tests, and a p value of <0.05 was considered significant.

### Results & Discussion

Heme is a highly hydrophobic molecule, and is mainly soluble in organic solvents or basic aqueous solutions. Therefore, in order to increase heme's aqueous solubility, colloidal stability, and anti-inflammatory therapeutic potential, we have exploited heme's ability to bind to the ubiquitous plasma protein human serum albumin (HSA) under basic conditions, and then covalently link poly (ethylene glycol) (PEG) to the surface of the heme-HSA conjugate to form PEGylated heme-HSA (PEG-heme-HSA). This strategy first increases the aqueous solubility of heme by first binding it to a carrier protein (HSA) that itself is soluble in aqueous solution, and then subsequently further improves the colloidal stability of heme-HSA by PEGylating it. Previously, our lab has stabilized various proteins such as apohemoglobin, and the annelid mega-Hb erythrocruorin via PEG surface conjugation leading to the increased colloidal stability of the PEGylated molecule. In this Example, we used thiol-maleimide chemistry to facilitate PEGylation of HSA. Surface lysine residues on HSA were reacted with iminothiolane to convert them into thiol groups, which were then reacted with PEG chains (MW 5000 Da) mono-functionalized with a maleimide reactive group. The conjugation reaction is site-specific and does not produce side products. The unreacted components were then removed via tangential flow filtration (TFF) using a 50 kDa hollow fiber (HF) filter. Therefore, in this study, we investigated heme incorporation into HSA, which was then PEGylated; as well as PEGylation of HSA, followed by heme incorporation on the biophysical properties of the final PEG-heme-HSA product.

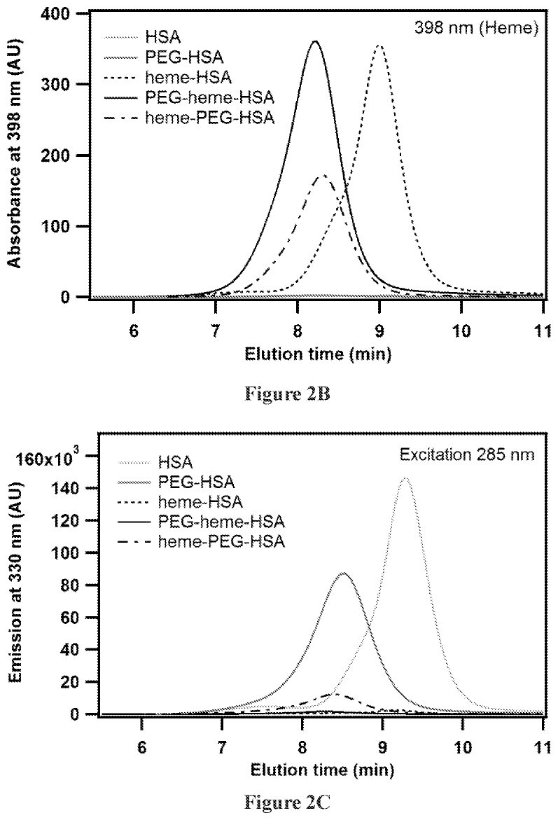

To analyze the incorporation of heme into HSA, SEC-HPLC analysis was performed as shown in FIGS. 2A-2C. The molecular weight (MW) of the protein was inversely proportional to the elution time from the column. HSA eluted from the column at 9.2 mins, whereas heme-HSA eluted earlier at ˜8.9 mins indicating successful heme incorporation. Conversion of elution time to MW using MW standards suggested that HSA had a MW of 66 kDa, whereas heme-HSA had a MW of ˜71 kDa. Furthermore, no peaks were observed at ˜13 mins, which corresponded to the presence of free heme (616 Da), and hence confirmed that TFF diafiltration and 0.2 μm syringe filtration was successful in removing any free heme or heme aggregates. Successful PEGylation of HSA was confirmed as the elution time of PEG-HSA was 8.4 mins, which corresponded to a MW of ˜130 kDa. Based on thiol quantification assays, we estimated that ˜5 PEG chains were attached to each HSA molecule resulting in a positive MW change of ˜25 kDa compared to HSA. The SEC-HPLC elution time to MW conversion, however, suggested that the MW change was ˜64 kDa. This discrepancy is expected as it has been reported previously that PEG conjugation exaggerates the MW change of conjugated proteins analyzed via SEC-HPLC due to the large hydration radius of the PEG conjugated molecules.

To incorporate heme in PEG-HSA, two approaches were used. First, heme was incorporated into previously PEGylated HSA. Second, heme was incorporated into HSA followed by PEG surface conjugation. The subsequent products were referred to as heme-PEG-HSA and PEG-heme-HSA, respectively. The elution time of heme-PEG-HSA was ˜8.2 mins, whereas it was ˜8.1 mins for PEG-heme-HSA corresponding to a MW of ˜140 kDa and ˜154 kDa respectively. This initially suggested that more heme was incorporated into HSA when PEGylation was performed after heme incorporation into HSA. Furthermore, for the same concentration of protein, the 398 nm absorbance peak area (detects heme) was ˜265 mAU*min for heme-HSA, 273 mAU*min for PEG-heme-HSA, and 146 mAU*min for heme-PEG-HSA. Note that no absorbance at 398 nm was observed for HSA and PEG-HSA, as none of these species had heme incorporation. Since the total protein concentration is directly proportional to the absorbance, this confirmed that less heme was incorporated into PEGylated HSA than unPEGylated HSA. This finding suggests that the steric hindrance caused by PEG chain conjugation on the surface of HSA allows less heme to be incorporated in the hydrophobic binding pockets of the protein. To further confirm these results, the excitation/emission spectra was measured at 285/330 nm respectively. Heme, among other metal-centered porphyrins, is known to quench the fluorescence of specific amino acid residues by dissipating the energy emitted in its structure. HSA and PEG-HSA exhibited significant fluorescence, whereas heme-HSA and PEG-heme-HSA had negligible fluorescence due to complete quenching of the fluorescence by the incorporated heme. However, heme-PEG-HSA had some residual fluorescence at 330 nm indicating only partial fluorescence quenching by the incorporated heme. These results confirmed that less heme was incorporated per HSA molecule when heme incorporation was performed after PEGylation of HSA. This further corroborated the idea that the PEG chains created steric hindrance on the outside surface of the HSA molecule allowing less heme to be incorporated. Hence, PEG-heme-HSA was identified as the optimal approach for synthesizing PEGylated heme-HSA, since HSA could incorporate more heme followed by PEGylation.

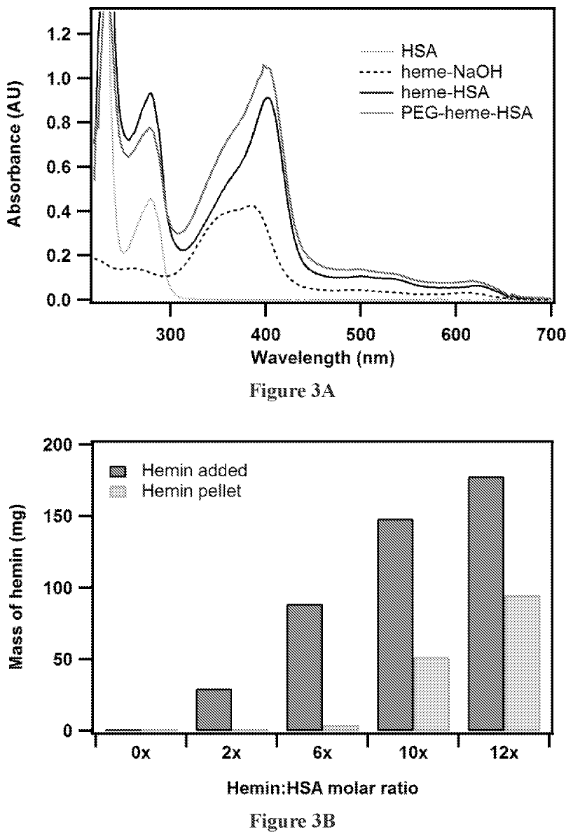

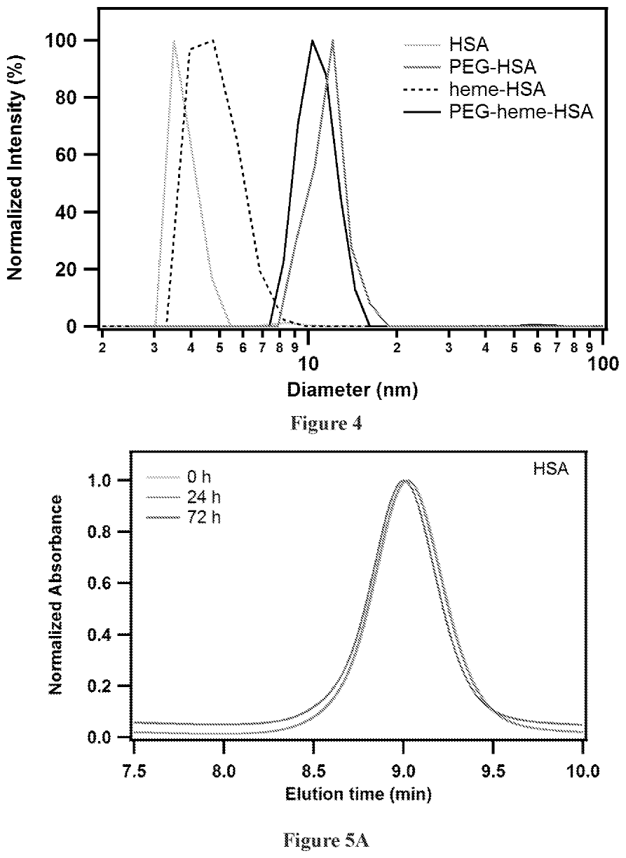

To determine unbound heme at the final product pH of 7.4, unbound heme was centrifuged to form a pellet, re-solubilized in NaOH, and quantified using UV-visible spectroscopy (FIGS. 3A-3B). During heme-HSA synthesis, molar ratios of heme:HSA ranging from 2:1 to 12:1 were investigated. However, it was observed that unbound heme was retained on the 0.2 μm syringe filter. Hence, samples were taken at multiple points during the synthesis process to quantify heme bound to HSA. On initially mixing heme-NaOH with HSA, there were no heme precipitate as all the heme was solubilized in the NaOH solution at pH 8.5. Similarly, no precipitate was observed after incubating the solution at 37° C. When the pH was balanced back to 7.4 using phosphoric acid, a significant pellet of heme was observed for higher heme:HSA molar ratios. Based on the difference between the initial mass of heme and the mass of the pellet, it was concluded that ˜5.8 heme molecules were bound to each HSA molecule at a 6:1 heme:HSA incubation reaction, 5.3 heme molecules were bound to each HSA molecule at a 10:1 heme-HSA incubation reaction, and 4.9 heme molecules were bound to each HSA molecule at a 12:1 heme-HSA incubation reaction. It is important to note that the maximum amount of heme bound to HSA remained almost constant with increasing heme:HSA molar ratio above 5:1 during synthesis. Hence, it was determined that a maximum of ˜5 heme molecules were bound to each HSA molecule at heme:HSA molar ratios >6:1. Previous studies have shown that HSA has a binding pocket that binds selectively to one heme molecule, hence, this study found that ˜4 additional heme molecules were non-selectively bound to HSA. It was interesting to note that no heme precipitated out of solution after the reaction, purification, and storage of heme-HSA in aqueous solution at 4° C. for over 1 week. Hence, we synthesized a heme-HSA molecule that has ˜5 hemes bound per HSA molecule that is stable in aqueous solution without loss or precipitation of heme.

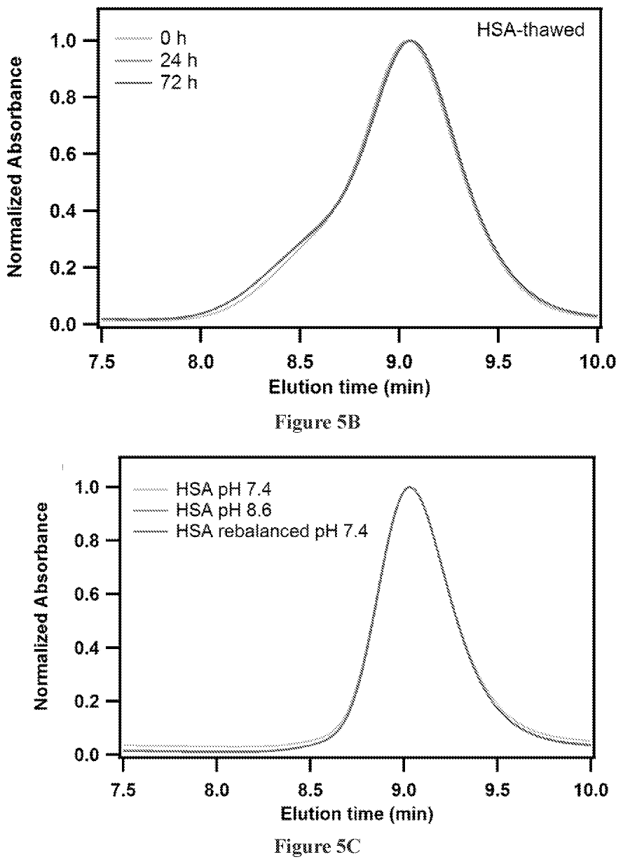

Dynamic light scattering (DLS) analysis was performed to determine the change in hydrodynamic diameter of HSA post PEGylation (FIG. 4). HSA by itself has a diameter of 3.8±0.4 nm, whereas after heme incorporation it increased to 5.1±2.2 nm, which was significantly different than HSA. The polydispersity index (PDI) of HSA went up from 0.1 to 0.3 post heme incorporation possibly due to the partial unfolding of the HSA structure under the basic pH conditions during heme incorporation. Even after the pH was adjusted to 7.4, it is likely that the HSA did not undergo complete refolding. This hypothesis was tested using circular dichroism (CD), and will be discussed later in the results and discussion section. Upon PEGylation of HSA, the hydrodynamic diameter increased to 13.1±0.6 nm confirming successful PEG surface conjugation. This was also statistically significant compared to HSA. Upon PEGylation of heme-HSA, the diameter increased from 5.1±1.2 nm to 11.9±1.4 nm, which was statistically significant, and the PDI was reduced from 0.3 to 0.2. Interestingly, there was no statistically significant difference in the diameter of PEG-HSA and PEG-heme-HSA. DLS analysis confirmed the results from SEC-HPLC that there was successful PEGylation of the HSA molecule before and after heme-incorporation. Furthermore, there were no other species detected in the DLS analysis denoting no free heme or aggregated heme particulates. PEG conjugation can result in similar diameter changes for proteins such as hemoglobin and apohemoglobin, which also improved the colloidal stability of the proteins in aqueous solution.

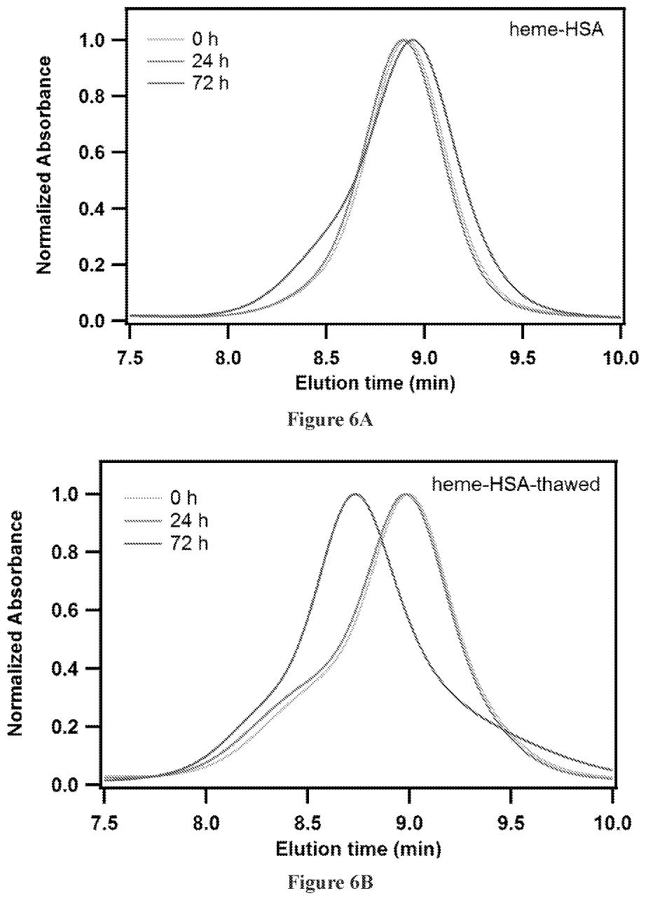

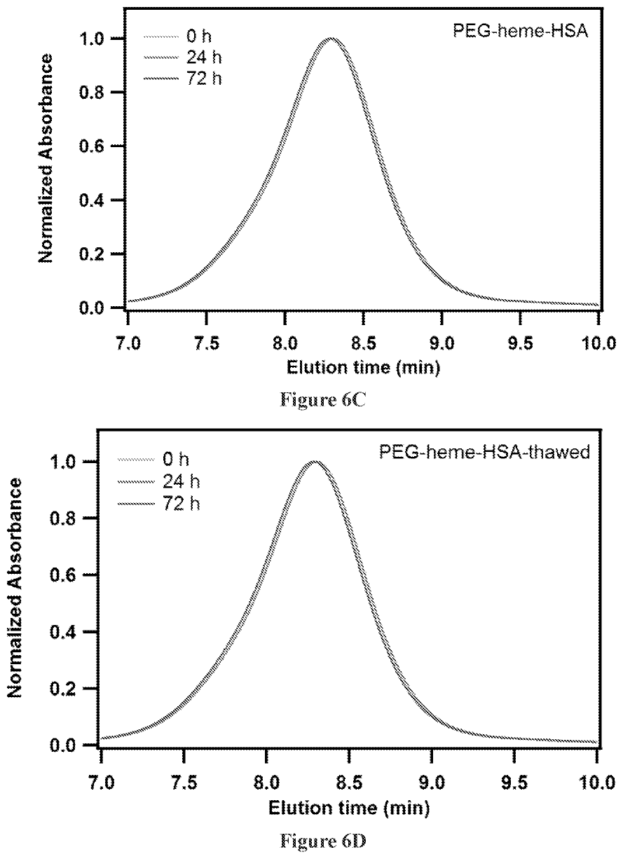

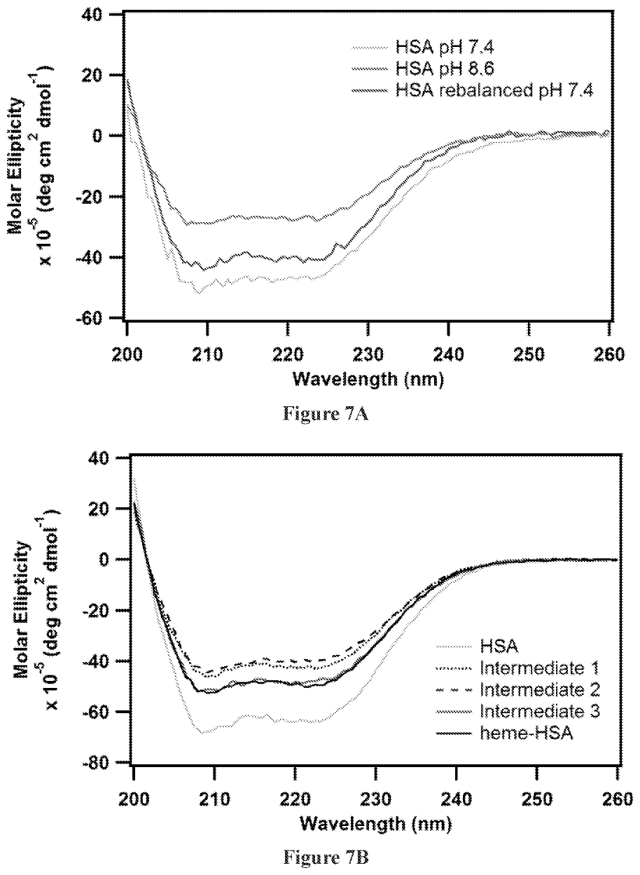

To further examine the colloidal stability of PEGylated heme-HSA in storage, all samples were tested under multiple storage conditions (FIGS. 5A-5C, 6A-6D). Samples were either stored at 4° C. for 72 hours with periodic SEC-HPLC measurements or frozen at −80° C., thawed, and then stored at 4° C. These conditions were selected to simulate storage of the product for future use in in vivo experiments that will evaluate the anti-inflammatory effects of heme. HSA did not exhibit any changes in quaternary structure after storage at 4° C. for 72 hours but showed ˜15% dimerization (elution time ˜8.4 mins) upon exposure to a single freeze-thaw cycle and subsequent storage at 4° C. for 72 hours. Furthermore, no change in the quaternary structure of HSA was observed in the pH rebalancing process. Upon storage of heme-HSA at 4° C., there was no change in the SEC-HPLC elution profile for ˜24 hours, but there was the appearance of dimeric species (elution time ˜8.4 mins) after 72 hours indicating protein aggregation. PEG-heme-HSA was very stable throughout the 72 hours storage period at 4° C., and did not show any change in the SEC-HPLC elution profile. Post one freeze-thaw cycle, heme-HSA yielded the appearance of >20% larger species (˜8.4 mins) at 0 hours, which increased to ˜90% larger species after 72 hours of storage at 4° C. Since we expect the HSA molecule to partially unfold under the basic conditions used for heme incorporation and refold after rebalancing to pH 7.4, its structural stability is compromised, which is further exaggerated following exposure to a single freeze thaw cycle. However, post PEGylation, the protein structure was stabilized, and we observed no change in the quaternary structure of PEGylated heme-HSA directly post a single freeze-thaw cycle, and after 72 hours of storage at 4° C. This further supports the increased colloidal stability of PEG-heme-HSA, and justified the use of PEG as a suitable molecule for heme-HSA surface conjugation and stabilization. Furthermore, it was important to note that no free heme aggregates were observed in all samples post exposure to a single freeze-thaw cycle.

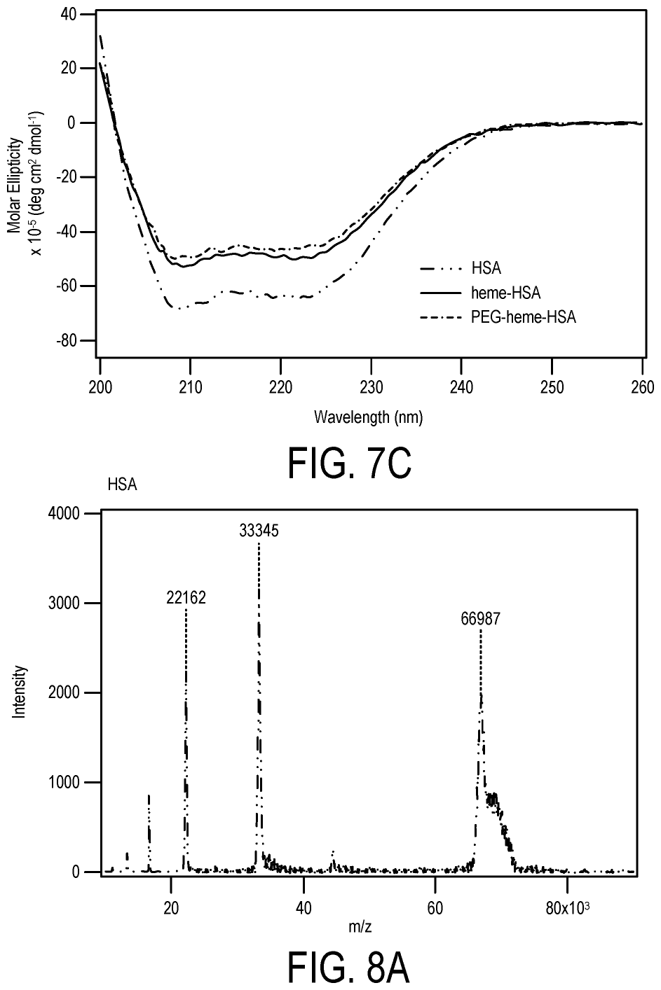

The change in α-helical content of HSA during heme-incorporation and post-modification was monitored using CD spectroscopy (FIGS. 7A-7C). The peak at 222 nm was used to monitor the change in α-helical content. The different intermediates are as follows: Intermediate 1—Heme-NaOH+HSA start of reaction pH 8.5; Intermediate 2—Heme-NaOH+HSA end of reaction pH 8.5; and Intermediate 3—Heme-NaOH+HSA pH balanced to 7.4. HSA has a hydrophobic pocket that selectively binds to one heme molecule. However, it also has non-selective heme-binding sites as confirmed via the heme pellet centrifugation quantification study. The α-helical content of heme-HSA intermediates 1 and 2 was reduced by ˜38% compared to HSA denoting partial unfolding of the HSA secondary structure under the basic conditions used for heme incorporation as hypothesized (FIG. 7B). The α-helical content of intermediate 3 and heme-HSA then increased to ˜80% of the original HSA α-helical content after balancing the pH back to 7.4 using phosphoric acid. It is interesting to observe that there was a permanent change in the secondary structure of HSA after heme incorporation as the α-helical content did not recover to that of the original HSA. This is consistent with HSA by itself which undergoes a ˜60% reduction in α-helical content after a pH change to 8.6, which does not fully recover after rebalancing the pH to 7.4 (FIG. 7A). After PEG-conjugation, the α-helical content reduced by ˜9% compared to heme-HSA indicating no major changes to the heme-HSA structure after the PEGylation process (FIG. 7C). These results confirmed the hypothesis that there was partial unfolding of the HSA molecule under the basic pH conditions conducive to heme incorporation, and subsequent partial refolding when the pH was adjusted back to 7.4.

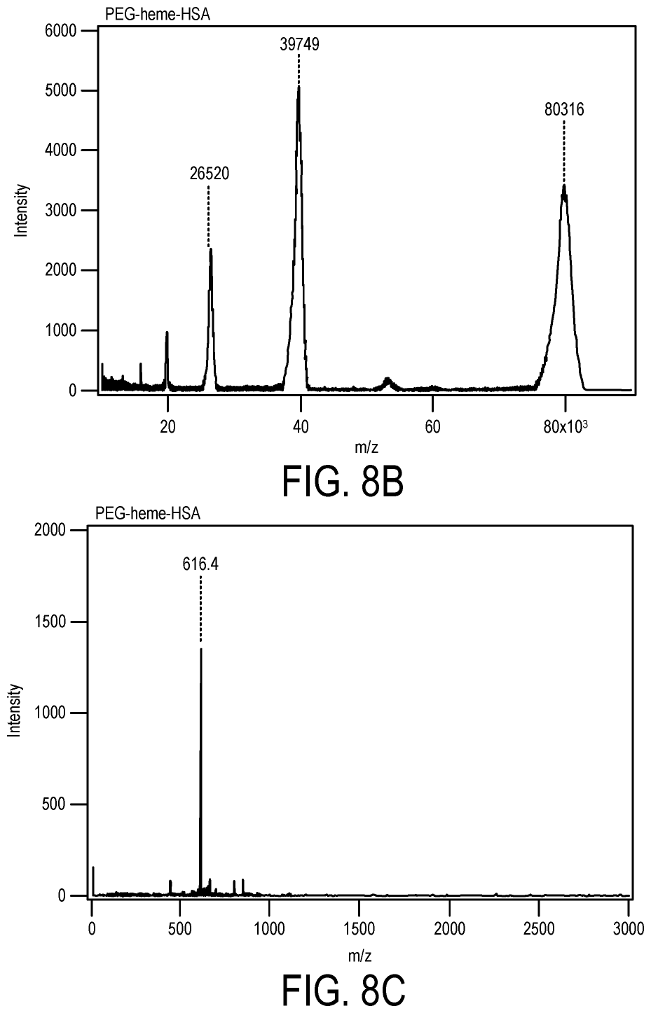

The change in the MW of HSA post PEGylation was verified using a MALDI-TOF MS analysis (FIGS. 8A-8C). The HSA control showed characteristic peaks at 66,000 m/z (+1 charged species), 33,000 m/z (+2 charged species), 22,000 (+3 charged species), etc. Upon PEGylation, the m/z shifted to ˜80,000 m/z for the +1 charged species confirming successful surface modification of HSA. There was also the presence of multiply charged peaks at 39,000 m/z, 26,000 m/z, etc. Furthermore, at the lowest m/z range, there was also the presence of a characteristic heme peak at 616 m/z denoting the presence of heme and its' incorporation into the protein. Comparing MALDI TOF MS analysis with SEC-HPLC MW estimates, the MW of PEG-heme-HSA was lower by ˜40 kDa. PEG chains can create a large hydration shell around the protein, which in turn exaggerates the MW estimates after SEC-HPLC analysis. This analysis combined with results from thiol quantification analysis concluded that ˜4 PEG chains were conjugated to the surface of each HSA molecule along with 1 specifically bound heme and ˜4 heme molecules non-specifically bound to the PEG-heme-HSA molecule.

In conclusion, we successfully incorporated heme into HSA followed by PEGylation to synthesize a colloidally-stable product that can be used in future studies to test the anti-inflammatory effects of heme in vitro and in vivo. The incorporation of heme into HSA followed by surface PEGylation was shown to incorporate more heme versus incorporating heme into already PEGylated HSA as confirmed by SEC-HPLC UV-visible analysis at 280 nm (to detect protein), 398 nm (to detect hemin), and fluorescence detection at 330 nm (to detect fluorescence quenching of incorporated heme). Furthermore, the increase in MW and hydrodynamic diameter of PEG-heme-HSA versus HSA was confirmed using SEC-HPLC and DLS analysis respectively. CD analysis confirmed the partial unfolding mechanism by which heme was incorporated into HSA, and MALDI-TOF MS analysis confirmed the change in MW before and after surface conjugation of heme-HSA with PEG. Finally, PEG-heme-HSA was found to be more colloidally-stable than heme-HSA, while storing the material at 4° C. and after exposure to a single freeze-thaw cycle and subsequent storage at 4° C. for 72 hours. Therefore, PEG-heme-HSA is a novel heme carrier that can be used in future in vivo experiments to test the potentially therapeutic anti-inflammatory effects of heme to treat disease.

### REFERENCES

(1) Nathan, C. Points of Control in Inflammation. Nature 2002, 420 (6917), 846-852. https://doi.org/10.1038/NATURE01320.
(2) Porto, B. N.; Alves, L. S.; Fernández, P. L.; Dutra, T. P.; Figueiredo, R. T.; Graça-Souza, A. V.; Bozza, M. T. Heme Induces Neutrophil Migration and Reactive Oxygen Species Generation through Signaling Pathways Characteristic of Chemotactic Receptors. J. Biol. Chem. 2007, 282 (33), 24430-24436. https://doi.org/10.1074/JBC.M703570200.
(3) Larsen, R.; Gozzelino, R.; Jeney, V.; Tokaji, L.; Bozza, F. A.; Japiassú, A. M.; Bonaparte, D.; Cavalcante, M. M.; Chora, A.; Ferreira, Â.; Marguti, I.; Cardoso, S.; Sepúlveda, N.; Smith, A.; Soares, M. P. A Central Role for Free Heme in the Pathogenesis of Severe Sepsis. Sci. Transl. Med. 2010, 2 (51). https://doi.org/10.1126/SCITRANSLMED.3001118.
(4) Vallelian, F.; Schaer, C. A.; Deuel, J. W.; Ingoglia, G.; Humar, R.; Buehler, P. W., Schaer, D. J. Revisiting the Putative Role of Heme as a Trigger of Inflammation. Pharmacol. Res. Perspect. 2018, 6 (2), e00392. https://doi.org/10.1002/prp2.392.
(5) Vijayan, V.; Wagener, F. A. D. T. G.; Immenschuh, S. The Macrophage Heme-Heme Oxygenase-1 System and Its Role in Inflammation. Biochem. Pharmacol. 2018, 153, 159-167. https://doi.org/10.1016/J.BCP.2018.02.010.
(6) vet Hanspeter Nägeli, med. Heme Drives Adaptive Hypoinflammation in Endothelial Cells. Valkova, Kristyna. Heme drives Adapt. hypoinflanimation Indothel. cells. 2018, Univ. Zurich, Vetsuisse Fac. 2018. https://doi.org/10.5167/UZH-165484.
(7) Janciauskiene, S.; Vijayan, V.; Immenschuh, S. TLR4 Signaling by Heme and the Role of Heme-Binding Blood Proteins. Front. Immunol. 2020, 11, 1964. https://doi.org/10.3389/FIMMU.2020.01964/BIBTEX.
(8) Stiebler, R., Hoang, A. N.; Egan, T. J.; Wright, D. W.; Oliveira, M. F. Increase on the Initial Soluble Heme Levels in Acidic Conditions Is an Important Mechanism for Spontaneous Heme Crystallization In Vitro. PLoS One 2010, 5 (9), 1-9. https://doi.org/10.1371/JOURNAL.PONE.0012694.
(9) Pires, I. S.; Savla, C.; Palmer, A. F. Poly(Ethylene Glycol) Surface-Conjugated Apohemoglobin as a Synthetic Heme Scavenger. Biomacromolecules 2020, 21(6), 2155-2164. https://doi.org/10.1021/acs.biomac.0c00141.
(10) Savla, C.; Palmer, A. F. Structural Stability and Biophysical Properties of the Mega-Protein Erythrocruorin Are Regulated by Polyethylene Glycol Surface Coverage. Biomacromolecules 2021, 22 (5), 2081-2093. https://doi.org/10.1021/acs.biomac.1c00196.
(11) Lombardo, M. E.; Araujo, L. S.; Ciccarelli, A. B.; Batlle, A. A Spectrophotometric Method for Estimating Hemin in Biological Systems. Anal. Biochem. 2005, 341 (2), 199-203. https://doi.org/10.1016/j.ab.2004.11.002.
(12) Akbarzadehlaleh, P.; Mirzaei, M.; Mashahdi-Keshtiban, M.; Shamsasenjan, K.; Heydari, H. PEGylated Human Serum Albumin: Review of PEGylation, Purification and Characterization Methods. Adv. Pharm. Bull. 2016, 6 (3), 309-317. https://doi.org/10.15171/apb.2016.043.
(13) Pace, C. N.; Vajdos, F.; Fee, L.; Grimsley, G.; Gray, T. How to Measure and Predict the Molar Absorption Coefficient of a Protein. Protein Si. 1995, 4 (11), 2411-2423. https://doi.org/10.1002/pro.5560041120.
(14) Winther, J. R.; Thorpe, C. Quantification of Thiols and Disulfides. Biochim. Biophys. Acta—Gen. Subj. 2014, 1840 (2), 838-846. https://doi.org/10.1016/j.bbagen.2013.03.031.
(15) Vandegriff, K. D.; Malavalli, A.; Mkrtchyan, G. M.; Spann, S. N.; Baker, D. A.; Winslow, R. M. Sites of Modification of Hemospan, a Poly(Ethylene Glycol)-Modified Human Hemoglobin for Use as an Oxygen Therapeutic. Bioconjug. Chem. 2008, 19 (11), 2163-2170. https://doi.org/10.1021/bc8002666.
(16) Mehtala, J. G.; Kulczar, C.; Lavan, M.; Knipp, G.; Wei, A. Cys34-PEGylated Human Serum Albumin for Drug Binding and Delivery. Bioconjug. Chem. 2015, 26 (5), 941-949. https://doi.org/10.1021/acs.bioconjchem.5b00143.
(17) Olson, K. R.; Gao, Y.; Arif, F.; Arora, K.; Patel, S.; DeLeon, E.; Straub, K. D. Innovative Methodology. Fluorescence Quenching by Metal Centered Porphyrins and Poryphyrin Enzymes. Am. J. Physiol.—Regul. Integr. Comp. Physiol. 2017, 313 (4), R340. https://doi.org/10.1152/AJPREGU.00202.2017.
(18) Wardell, M.; Wang, Z.; Ho, J. X.; Robert, J.; Ruker, F.; Ruble, J.; Carter, D. C. The Atomic Structure of Human Methemalbumin at 1.9 Å. Biochem. Biophys. Res. Commun. 2002, 291 (4), 813-819. https://doi.org/10.1006/bbrc.2002.6540.
(19) Hu, T.; Prabhakaran, M.; Acharya, S. A.; Manjula, B. N. Influence of the Chemistry of Conjugation of Poly(Ethylene Glycol) to Hb on the Oxygen-Binding and Solution Properties of the PEG-Hb Conjugate. Biochem. J. 2005, 392 (3), 555-564. https://doi.org/10.1042/BJ20050663.
(20) Smani, Y. Hemospan: A Hemoglobin-Based Oxygen Carrier for Potential Use as a Blood Substitute and for the Potential Treatment of Critical Limb Ischemia. Curr. Opin. Investig. Drugs 2008, 9 (9), 1009-1019.
(21) Lawrence, P. B.; Price, J. L. How PEGylation Influences Protein Conformational Stability. Curr. Opin. Chem. Biol. 2016, 34, 88-94. https://doi.org/10.1016/j.cbpa.2016.08.006.

The compositions and methods of the appended claims are not limited in scope by the specific compositions and methods described herein, which are intended as illustrations of a few aspects of the claims. Any compositions and methods that are functionally equivalent are intended to fall within the scope of the claims. Various modifications of the compositions and methods in addition to those shown and described herein are intended to fall within the scope of the appended claims. Further, while only certain representative compounds, components, compositions, and method steps disclosed herein are specifically described, other combinations of the compounds, components, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A method for forming a colloidally stable heme-albumin complex, the method comprising:
contacting albumin with heme under conditions effective to form a heme-albumin complex;
conjugating one or more hydrophilic polymers to the heme-albumin complex to form a crude colloidally stable heme-albumin complex; and
filtering the crude colloidally stable heme-albumin complex by ultrafiltration against a filtration membrane, thereby forming a retentate fraction comprising the colloidally stable heme-albumin complex and a permeate fraction comprising low molecular weight contaminants.

2. The method of claim 1, wherein the colloidally stable heme-albumin complex is substantially free of free heme, aggregated heme particulates, unconjugated hydrophilic polymers, and other low molecular weight contaminants.

3. The method of any of claims 1-2, wherein the colloidally stable heme-albumin complex comprises less than 1% by weight free heme, aggregated heme particulates, unconjugated hydrophilic polymers, and other low molecular weight contaminants having a molecular weight of less than 50 kDa, such as less than 0.5% by weight free heme, aggregated heme particulates, unconjugated hydrophilic polymers, and other low molecular weight contaminants having a molecular weight of less than 50 kDa, or less than 0.1% by weight free heme, aggregated heme particulates, unconjugated hydrophilic polymers, and other low molecular weight contaminants having a molecular weight of less than 50 kDa.

4. The method of any of claims 1-3, wherein contacting albumin with heme under conditions effective to form a heme-albumin complex comprises incubating the albumin with heme at a pH of 8 or more, such as a pH of from 8 to 10.

5. The method of claim 4, wherein the heme and the albumin are present at a molar ratio of heme:albumin of at least 5:1, such as a molar ratio of from 5:1 to 15:1, such as from 5:1 to 12:1, from 5:1 to 10:1, or from 5:1 to 8:1.

6. The method of any of claims 4-5, wherein contacting albumin with heme under conditions effective to form a heme-albumin complex further comprises neutralizing the heme-albumin complex.

7. The method of any of claims 1-6, wherein the filtration membrane has a filter rated for removing solutes having a molecular weight of from 1 to 50 kDa.

8. The method of any of claims 1-7, wherein filtering crude colloidally stable heme-albumin complex by ultrafiltration comprises filtering for at least 3 diafiltrations cycles, such as at least 4 diafiltration cycles, at least 5 diafiltration cycles, at least 6 diafiltration cycles, at least 7 diafiltrations cycle, at least 8 diafiltration cycles, at least 9 diafiltrations cycle, or at least 10 diafiltration cycles.

9. The method of any one of claims 1-8, wherein the ultrafiltration comprises tangential flow filtration.

10. The method of any one of claims 1-9, wherein the one or more hydrophilic polymers comprise poly(lactide), poly(glycolide), a poly(orthoesters), a poly(caprolactone), polylysine, poly(ethylene imine), poly(acrylic acid), a poly(urethane), a poly(anhydride), a poly(ester), poly(trimethylene carbonate), poly(ethyleneimine), a poly(acrylic acid), a poly(urethane), a poly(beta amino ester), a poly(alkylene oxide) such as polyethylene glycol (PEG), a zwitterionic polymer, a copolymer thereof, or a blend thereof.

11. The method of any one of claims 1-10, wherein conjugating the one or more hydrophilic polymers to the heme-albumin complex to form the crude colloidally stable heme-albumin complex comprises contacting the heme-albumin complex with a derivatized hydrophilic polymer under conditions permitting formation of a covalent bond between the hydrophilic polymer and the heme-albumin complex so as to form the crude colloidally stable heme-albumin complex.

12. The method of any one of claims 1-10, wherein the one or more hydrophilic polymers comprise polyethylene glycol (PEG).

13. The method of claim 12, wherein conjugating the one or more PEG polymers to the heme-albumin complex to form the crude colloidally stable heme-albumin complex comprises contacting the heme-albumin complex with a derivatized PEG polymer under conditions permitting formation of a covalent bond between the PEG polymer and the heme-albumin complex so as to form the crude colloidally stable heme-albumin complex.

14. The method of claim 13, wherein the derivatized PEG comprises succinimidyl-PEG, cyanuric chloride-PEG, or maleimide-PEG.

15. The method of any of claims 13-14, wherein conjugating the one or more PEG polymers to the heme-albumin complex to form the crude colloidally stable heme-albumin complex further comprises contacting the heme-albumin complex with a thiolation reagent, such as 2-iminothiolane hydrochloride, to introduce thiol moieties that react with the derivatized PEG.

16. The method of any of claims 1-15, wherein the one or more hydrophilic polymers each have a molecular weight of from 200 Da to 20,000 Da, such as from 1,000 Da to 10,000 Da, or from 3,000 Da to 6,000 Da, or about 5,000 Da.

17. The method of any of claims 1-16, wherein the method further comprises filtering the heme-albumin complex by ultrafiltration against a filtration membrane prior to conjugating one or more hydrophilic polymers to the heme-albumin complex, thereby forming a retentate fraction comprising the heme-albumin complex and a permeate fraction comprising low molecular weight contaminants.

18. The method of claim 17, wherein following filtration, the heme-albumin complex is substantially free of free heme, aggregated heme particulates, and other low molecular weight contaminants.

19. The method of any of claims 17-18, wherein following filtration, the heme-albumin complex comprises less than 1% by weight free heme, aggregated heme particulates, and other low molecular weight contaminants having a molecular weight of less than 50 kDa, such as less than 0.5% by weight free heme, aggregated heme particulates, and other low molecular weight contaminants having a molecular weight of less than 50 kDa, or less than 0.1% by weight free heme, aggregated heme particulates, and other low molecular weight contaminants having a molecular weight of less than 50 kDa.

20. The method of any of claims 1-19, wherein the colloidally stable heme-albumin complex comprises five heme molecules associated with one albumin protein.

21. The method of any of claims 1-20, wherein the colloidally stable heme-albumin complex is stable in aqueous solution after 72 hours of storage at 4° C. without loss or precipitation of heme.

22. The method of any of claims 1-21, wherein the colloidally stable heme-albumin complex exhibits a hydrodynamic diameter of from about 10 nm to about 15 nm, as determined by dynamic light scattering (DLS).

23. The method of any of claims 1-22, wherein the colloidally stable heme-albumin complex comprises from 3 to 10 hydrophilic polymer chains conjugated to the heme-albumin complex, as determined by thiol quantification.

24. A method for forming a colloidally stable heme-albumin complex, the method comprising:
conjugating one or more hydrophilic polymers to albumin to form polymer-modified albumin;
contacting the polymer-modified albumin with heme under conditions effective to form a crude colloidally stable heme-albumin complex; and
filtering the crude colloidally stable heme-albumin complex by ultrafiltration against a filtration membrane, thereby forming a retentate fraction comprising the colloidally stable heme-albumin complex and a permeate fraction comprising low molecular weight contaminants.

25. The method of claim 24, wherein the colloidally stable heme-albumin complex is substantially free of free heme, aggregated heme particulates, unconjugated hydrophilic polymers, and other low molecular weight contaminants.

26. The method of any of claims 24-25, wherein the colloidally stable heme-albumin complex comprises less than 1% by weight free heme, aggregated heme particulates, unconjugated hydrophilic polymers, and other low molecular weight contaminants having a molecular weight of less than 50 kDa, such as less than 0.5% by weight free heme, aggregated heme particulates, unconjugated hydrophilic polymers, and other low molecular weight contaminants having a molecular weight of less than 50 kDa, or less than 0.1% by weight free heme, aggregated heme particulates, unconjugated hydrophilic polymers, and other low molecular weight contaminants having a molecular weight of less than 50 kDa.

27. The method of any of claims 24-26, wherein contacting the polymer-modified albumin with heme under conditions effective to form a crude colloidally stable heme-albumin complex comprises incubating the polymer-modified albumin with heme at a pH of 8 or more, such as a pH of from 8 to 10.

28. The method of claim 27, wherein the heme and the polymer-modified albumin are present at a molar ratio of heme:polymer-modified albumin of at least 5:1, such as a molar ratio of from 5:1 to 15:1, such as from 5:1 to 12:1, from 5:1 to 10:1, or from 5:1 to 8:1.

29. The method of any of claims 27-28, wherein contacting polymer-modified albumin with heme under conditions effective to form a heme-albumin complex further comprises neutralizing the crude colloidally stable heme-albumin complex.

30. The method of any of claims 24-29, wherein the filtration membrane has a filter rated for removing solutes having a molecular weight of from 1 to 50 kDa.

31. The method of any of claims 24-30, wherein filtering crude colloidally stable heme-albumin complex by ultrafiltration comprises filtering for at least 3 diafiltrations cycles, such as at least 4 diafiltration cycles, at least 5 diafiltration cycles, at least 6 diafiltration cycles, at least 7 diafiltrations cycle, at least 8 diafiltration cycles, at least 9 diafiltrations cycle, or at least 10 diafiltration cycles.

32. The method of any one of claims 24-31, wherein the ultrafiltration comprises tangential flow filtration.

33. The method of any one of claims 24-32, wherein the one or more hydrophilic polymers comprise poly(lactide), poly(glycolide), a poly(orthoesters), a poly(caprolactone), polylysine, poly(ethylene imine), poly(acrylic acid), a poly(urethane), a poly(anhydride), a poly(ester), poly(trimethylene carbonate), poly(ethyleneimine), a poly(acrylic acid), a poly(urethane), a poly(beta amino ester), a poly(alkylene oxide) such as polyethylene glycol (PEG), a zwitterionic polymer, a copolymer thereof, or a blend thereof.

34. The method of any one of claims 24-33, wherein conjugating the one or more hydrophilic polymers to the albumin to form the polymer-modified albumin complex comprises contacting the albumin with a derivatized hydrophilic polymer under conditions permitting formation of a covalent bond between the hydrophilic polymer and the albumin so as to form the polymer-modified albumin.

35. The method of any one of claims 24-34, wherein the one or more hydrophilic polymers comprise polyethylene glycol (PEG).

36. The method of claim 35, wherein conjugating the one or more PEG polymers to the albumin to form PEGylated albumin comprises contacting the albumin with a derivatized PEG polymer under conditions permitting formation of a covalent bond between the PEG polymer and the albumin so as to form the PEGylated albumin.

37. The method of claim 36, wherein the derivatized PEG comprises succinimidyl-PEG, cyanuric chloride-PEG, or maleimide-PEG.

38. The method of any of claims 36-37, wherein conjugating the one or more PEG polymers to the albumin to form the PEGylated albumin further comprises contacting the albumin with a thiolation reagent, such as 2-iminothiolane hydrochloride, to introduce thiol moieties that react with the derivatized PEG.

39. The method of any of claims 24-38, wherein the one or more hydrophilic polymers each have a molecular weight of from 200 Da to 20,000 Da, such as from 1,000 Da to 10,000 Da, or from 3,000 Da to 6,000 Da, or about 5,000 Da.

40. The method of any of claims 24-39, wherein the method further comprises filtering the polymer-modified albumin by ultrafiltration against a filtration membrane prior to contacting the polymer-modified albumin with heme under conditions effective to form a crude colloidally stable heme-albumin complex, thereby forming a retentate fraction comprising the polymer-modified albumin and a permeate fraction comprising low molecular weight contaminants.

41. The method of claim 40, wherein following filtration, the polymer-modified albumin is substantially free of unconjugated hydrophilic polymers and other low molecular weight contaminants.

42. The method of any of claims 40-41, wherein following filtration, the polymer-modified albumin comprises less than 1% by weight free unconjugated hydrophilic polymers and other low molecular weight contaminants having a molecular weight of less than 50 kDa, such as less than 0.5% by weight unconjugated hydrophilic polymers and other low molecular weight contaminants having a molecular weight of less than 50 kDa, or less than 0.1% by weight unconjugated hydrophilic polymers and other low molecular weight contaminants having a molecular weight of less than 50 kDa.

43. The method of any of claims 24-42, wherein the colloidally stable heme-albumin complex comprises five heme molecules associated with one albumin protein.

44. The method of any of claims 24-43, wherein the colloidally stable heme-albumin complex is stable in aqueous solution after 72 hours of storage at 4° C. without loss or precipitation of heme.

45. The method of any of claims 24-44, wherein the colloidally stable heme-albumin complex exhibits a hydrodynamic diameter of from about 10 nm to about 15 nm, as determined by dynamic light scattering (DLS).

46. The method of any of claims 24-45, wherein the colloidally stable heme-albumin complex comprises from 3 to 10 hydrophilic polymer chains conjugated to the heme-albumin complex, as determined by thiol quantification.

47. The method of any of claims 1-46, wherein the albumin comprises a serum albumin, such as human serum albumin or recombinant human serum albumin.

48. A composition comprising a colloidally stable heme-albumin complex dissolved or dispersed in an aqueous carrier, wherein the colloidally stable heme-albumin complex comprising from three to six heme molecules non-covalently associated with an albumin protein, and a plurality of hydrophilic polymers conjugated to the albumin protein.

49. The composition of claim 48, wherein the composition is substantially free of free heme, aggregated heme particulates, unconjugated hydrophilic polymers, and other low molecular weight contaminants.

50. The composition of any of claims 48-49, wherein the composition comprises less than 1% by weight free heme, aggregated heme particulates, unconjugated hydrophilic polymers, and other low molecular weight contaminants having a molecular weight of less than 50 kDa, such as less than 0.5% by weight free heme, aggregated heme particulates, unconjugated hydrophilic polymers, and other low molecular weight contaminants having a molecular weight of less than 50 kDa, or less than 0.1% by weight free heme, aggregated heme particulates, unconjugated hydrophilic polymers, and other low molecular weight contaminants having a molecular weight of less than 50 kDa.

51. The composition of any of claims 48-50, wherein the one or more hydrophilic polymers comprise poly(lactide), poly(glycolide), a poly(orthoesters), a poly(caprolactone), polylysine, poly(ethylene imine), poly(acrylic acid), a poly(urethane), a poly(anhydride), a poly(ester), poly(trimethylene carbonate), poly(ethyleneimine), a poly(acrylic acid), a poly(urethane), a poly(beta amino ester), a poly(alkylene oxide) such as polyethylene glycol (PEG), a zwitterionic polymer, a copolymer thereof, or a blend thereof.

52. The composition of any of claims 48-51, wherein the one or more hydrophilic polymers each have a molecular weight of from 200 Da to 20,000 Da, such as from 1,000 Da to 10,000 Da, or from 3,000 Da to 6,000 Da, or about 5,000 Da.

53. The composition of any of claims 48-52, wherein the one or more hydrophilic polymers comprise polyethylene glycol (PEG).

54. The composition of any of claims 48-53, wherein the colloidally stable heme-albumin complex comprises five heme molecules associated with one albumin protein.

55. The composition of any of claims 48-54, wherein the colloidally stable heme-albumin complex is stable in aqueous solution after 72 hours of storage at 4° C. without loss or precipitation of heme.

56. The composition of any of claims 48-55, wherein the colloidally stable heme-albumin complex exhibits a hydrodynamic diameter of from about 10 nm to about 15 nm, as determined by dynamic light scattering (DLS).

57. The composition of any of claims 48-56, wherein the colloidally stable heme-albumin complex comprises from 3 to 10 hydrophilic polymer chains conjugated to the heme-albumin complex, as determined by thiol quantification.

58. The composition of any of claims 48-57, wherein the albumin comprises a serum albumin, such as human serum albumin or recombinant human serum albumin.

59. A method of treating an ophthalmological disorder in a subject in need thereof comprising contacting the eye of the subject a therapeutically effective amount of the composition of any of claims 48-58.

60. The method of claim 59, wherein the composition further comprises an additional active agent.

61. The method of claim 60, wherein the additional active agent comprises an ophthalmic drug, such as an anti-glaucoma agent, an anti-angiogenesis agent, an anti-vascular endothelial growth factor (VEGF) agent, an anti-infective agent, an anti-inflammatory agent, a growth factor, an immunosuppressant agent, an anti-allergic agent, or any combinations thereof.

62. The method of any of claims 59-61, wherein the ophthalmological disorder is acute macular neuroretinopathy; Behcet's disease; neovascularization, including choroidal neovascularization; diabetic uveitis; histoplasmosis; infections, such as fungal or viral-caused infections; macular degeneration, such as acute macular degeneration (AMD), including wet AMD, non-exudative AMD and exudative AMD; retinal degenerative diseases such as geographic atrophy; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic ophthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, nonretinopathy diabetic retinal dysfunction, retinitis pigmentosa, a cancer, and glaucoma.

63. The method of claim 62, wherein the ophthalmological disorder is AMD, such as dry AMD.

64. The method of any one of claims 59-63, wherein contacting the eye of the subject comprises topically applying the composition to the eye of the subject.

65. The method of any one of claims 59-63, wherein contacting the eye of the subject comprises injecting the composition into the eye of the subject.

66. The method of claim 65, wherein injecting into the eye of the subject comprises injecting the ocular therapeutic composition into the vitreous chamber of the eye.

67. The method of any one of claims 65-66, wherein injecting into the eye of the subject comprises an intravitreal injection, a subconjunctival injection, a subtenon injection, a retrobulbar injection, or a suprachoroidal injection.

68. A method of reducing inflammation in a subject, the method comprising administering a therapeutically effective amount of the composition of any of claims 48-58 to the subject.

69. A colloidally stable heme-albumin complex prepared by the method of any of claims 1-47.
